(19) United States
(12) Patent Application Publication
(10) Pub. No.: US 2021/0395396 A1
(43) Pub. Date: Dec. 23, 2021

(54) HER2-BINDING TETRAMERIC POLYPEPTIDES

(71) Applicant: UNIVERSITÄT ZÜRICH, Zurich (CH)

(72) Inventors: Florian KAST, Zurich (CH); Martin SCHWILL, Zurich (CH); Annemarie HONEGGER, Zurich (CH); Jakob STÜBER, München (DE); Rastislav TAMASKOVIC, Rheinfelden (CH); Andreas PLÜCKTHUN, Zurich (CH)

(73) Assignee: UNIVERSITÄT ZÜRICH, Zurich (CH)

(21) Appl. No.: 17/282,781

(22) PCT Filed: Oct. 8, 2019
(86) PCT No.: PCT/EP2019/077147

§ 371 Date: Apr. 5, 2021

Related U.S. Application Data
(63) Continuation-in-part of application No. 16/153,857, filed on Oct. 8, 2018.
Application No. 16/153,857 is a continuation-in-part of application No. 14/430,224, filed on Mar. 22, 2015, now Pat. No. 10,093,740.

(30) Foreign Application Priority Data
Oct. 15, 2012 (EP) 12188598.2
Nov. 7, 2012 (EP) 12191673.8
Nov. 13, 2012 (EP) 12192465.8
Sep. 24, 2013 (EP) 13185724.5
Mar. 12, 2019 (EP) 19162408.9
Mar. 26, 2019 (EP) 19165362.5
Apr. 30, 2019 (EP) 19172075.4

(51) Int. Cl. C07K 16/46; C07K 16/32

(52) U.S. Cl. CPC C07K 16/46; C07K 16/32; C07K 2317/66; C07K 2319/80; C07K 2317/56; C07K 2317/24

(57) ABSTRACT

The invention relates to a tetrameric polypeptide comprising a first polypeptide chain comprising a first VL antigen binding domain and a first CL constant domain, a second polypeptide chain comprising a first VH antigen binding domain, a first CH1 constant domain, a first CH2 constant domain and a first CH3 constant domain, a first ligand binding to a HER2 D4 epitope linked to the N-terminus of said first VL antigen binding domain or said first VH antigen binding domain by a first interdomain amino acid linker, a third polypeptide chain comprising a second VL antigen binding domain and a second CL constant domain, a fourth polypeptide chain comprising a second VH antigen binding domain, a second CH1 constant domain, a second CH2 constant domain and a second CH3 constant domain and a third ligand binding to a HER2 D4 epitope linked to the N-terminus of said second VL antigen binding domain or said second VH antigen binding domain by a second interdomain amino acid linker, wherein the VL antigen binding domains and the VH antigen binding domains together constitute a second ligand and a fourth ligand binding to a HER2 D1 epitope. The invention further relates to the tetrameric polypeptide for use in a method for the prevention or treatment of a malignant neoplastic disease, an isolated nucleic acid and a host cell for expression of the polypeptide and a method for obtaining the polypeptide.

The present invention relates to a tetrameric polypeptide having two binding sites to HER2 epitope D1 and two binding sites to HER2 epitope D4.

### BACKGROUND OF THE INVENTION

The members of the HER family of receptor tyrosine kinases are important mediators of cell growth, differentiation, migration and survival. The receptor family includes four distinct members including epidermal growth factor receptor (EGFR, ErbB1, or HER1), HER2 (ErbB2 or p185<neu>), HER3 (ErbB3) and HER4 (ErbB4). The members of the EGFR family are closely related single-chain modular glycoproteins with an extracellular ligand binding region, a single transmembrane domain and an intracellular tyrosine kinase, followed by specific phosphorylation sites which are important for the docking of downstream signaling proteins.

The extracellular regions of the HER receptor family contain two homologous ligand binding domains (domains 1 and 3) and two cysteine-rich domains (domains 2 and 4), which are important for receptor dimerization. In the absence of a ligand, HER receptors normally exist as inactive monomers, known as the “tethered” structure, which is characterized by close interaction of domain 2 and 4. Ligand binding to the extracellular domain initiates a conformational rearrangement, exposing the dimerization domains 2 and 4. Therefore, binding of growth factors to HER receptors induces conformational changes that allow receptor dimerization. After extracellular receptor dimerization, transmembrane helices switch to an active conformation that enables the intracellular kinase domains to trans-auto-phosphorylate each other. This phosphorylation event allows the recruitment of specific downstream signaling proteins.

Epidermal Growth factor receptor 1, (EGFR), has been causally implicated in human malignancy. In particular, increased expression of EGFR has been observed in breast, bladder, lung, head, neck and stomach cancer as well as glioblastomas.

Human epidermal growth factor receptor 2 (HER2, also known as ErbB2 or Neu; UniProtKB/Swiss-Prot No. P04626) consists of 1233 amino acids and is structurally similar to EGFR, with an extracellular domain consisting of four subdomains 1-4, a transmembrane domain, a juxtamembrane domain, an intracellular cytoplasmic tyrosine kinase and a regulatory C-terminal region. The structure of HER2′s extracellular region is different in important aspects from the other EGF receptors, however. In the other EGF receptors, in a non-activated state, domain 2 binds to domain 4. Upon binding to domains 1 and 3, the activating growth factor (ligand) selects and stabilizes a conformation that allows a dimerization arm to extend from domain 2 to interact with an ErbB dimer partner. HER2, on the other hand, has a fixed conformation that resembles the ligand-activated state of the other receptor members: the domain 2-4 interaction is absent and the dimerization loop in domain 2 is continuously exposed. HER2 is activated via formation of heterodimeric complexes with other ErbB family members and thereby indirectly regulated by EGFR and HER3 ligands. HER2 is the preferred heterodimerization partner of the three other ErbB receptors, enhancing the affinity of the other ErbB receptors for their ligands by slowing down the rate of ligand-receptor complex dissociation, whereby HER2 enhances and prolongs signaling.

An excess of HER2 on the cell surface causes transformation of epithelial cells from multiple tissues. Amplification of the human homolog of the neu gene (also known as HER2) is observed in breast and ovarian cancers and correlates with a poor prognosis (U.S. Pat. No. 4,968,603). Overexpression of HER2 has also been observed in other carcinomas including carcinomas of the stomach, endometrium, salivary gland, lung, kidney, colon, thyroid, pancreas and bladder.

Antibodies Targeting HER2

Drebin and colleagues have raised antibodies against the rat neu gene product, p185<neu>disclosed in US6,733,752.

Hudziak et al. (1989, Mol. Cell. Biol. 9(3), 1165-1172) describe the generation of a panel of HER2 antibodies which were characterized using the human breast tumor cell line SkBr-3. Using a cell proliferation assay, maximum inhibition was obtained with an antibody called 4D5. The antibody 4D5 was further found to sensitize HER2-overexpressing breast tumor cell lines to the cytotoxic effects of TNF-[alpha]; see also U.S. Pat. No. 5,677,171. A recombinant humanized version of the murine HER2 antibody 4D5 (huMAb4D5-8, rhuMAb HER2, trastuzumab or HERCEPTIN; US 5,821,337) is clinically active in patients with HER2-overexpressing metastatic breast cancers that have received extensive prior anti-cancer therapy. Herceptin is approved in combination with chemotherapy for use in patients with HER2-positive metastatic stomach (gastric) cancer.

Herceptin is widely used for the treatment of patients with early as well as metastatic breast cancer whose tumors overexpress HER2 protein and/or have HER2 gene amplification. The treatment of breast cancer patients with Herceptin/trastuzumab is, for example, recommended and now routine for patients having HER2-positive disease; see US 2002/0064785, US 2003/0170234A1, US2003/0134344 and US 2003/0147884. The prior art thus focuses on the eligibility of breast cancer patients for trastuzumab/Herceptin therapy based on a high HER2 protein expression level (e.g. defined as HER2(3+) by immunohistochemistry (IHC)). HER2-positive disease in breast cancer is defined to be present if a high HER2 (protein) expression level is detected by immunohistochemical methods (e.g. HER2 (+++) or as HER2 gene amplification (e.g. a HER2 gene copy number higher than 4 copies of the HER2 gene per tumor cell) or both, found in samples obtained from the patients such as breast tissue biopsies or breast tissue resections or in tissue derived from metastatic sites. One frequently applied method for detecting HER2 overexpression and amplification at the gene level is fluorescence in situ hybridization (FISH), which is also described in US 2003/0152987.

Pertuzumab, a humanized antibody, is the first of a new class of agents known as HER dimerization inhibitors (HDIs). Pertuzumab binds to HER2 at its dimerization domain, thereby inhibiting its ability to form active heterodimer receptor complexes, thus blocking the downstream signal cascade that ultimately results in cell growth and division. Pertuzumab is directed against the extracellular domain 2 of HER2. In contrast to trastuzumab, which acts by binding to domain 4 of HER2, pertuzumab is a HER dimerization inhibitor which inhibits dimerization of HER2 with HER3 and the other members of the EGFR receptor family in the presence of the respective activating ligands. By blocking complex formation, pertuzumab prevents the growth-stimulatory effects and cell survival signals activated by ligands of HER1, HER3 and HER4. Pertuzumab has been approved by the FDA under the name Perjeta for treatment in combination with trastuzumab and docetaxel for patients with HER2-positive metastatic breast cancer, who have not received prior anti-HER2 therapy or chemotherapy for metastatic disease. Pertuzumab is a fully humanized recombinant monoclonal antibody based on the human IgG1([kappa]) framework sequences. Patent publications concerning pertuzumab and selection of patients for therapy therewith include: US20060073143A1; US20030086924; US20040013667A1, and US20040106161A1.

For trastuzumab, while known to show clinical benefits in terms of e.g. prolonged survival in combination with chemotherapy compared to chemotherapy alone, a majority of HER2 positive breast cancer patients were nevertheless found to be non-responders (45% overall response rate for Herceptin +chemotherapy vs. 29% for chemotherapy alone).

Thus, while monoclonal antibody therapy directed against HER2 has been shown to provide improved treatment in e.g. metastatic breast cancers that overexpress HER2, there is still considerable room for improvement.

Non-Antibody Scaffolds Targeting HER2

Alternative targeting proteins have been proposed recently, which are more diverse in molecular structure than human immunoglobulin-derived antibody fragments and antibody-derived constructs and formats, and thus allow additional molecular formats by creating heterodimeric and multimeric assemblies, leading to new biological functions. A number of such targeting proteins have been described (reviewed in (Binz et al., 2005, Nat. Biotech, 23, 1257-1268)). Non-limiting examples of such targeting proteins are camelid antibodies, protein scaffolds derived from protein A domains (termed “Affibodies”, Affibody AB), tendamistat (an alpha-amylase inhibitor, a 74 amino acid beta-sheet protein from Streptomyces tendae), fibronectin, lipocalin (“Anticalins”, Pieris), T-cell receptors, ankyrins (designed ankyrin repeat proteins termed “DARPins”, Univ. Zurich and Molecular Partners; see US20120142611), A-domains of several receptors (“Avimers”, Avidia) and PDZ domains, fibronectin domains (FN3) (“Adnectins”, Adnexus), consensus fibronectin domains (“Centyrins”, Centyrex/Johnson&Johnson) and Ubiquitin (“Affilins”, SCIL Proteins) and knottins (Moore and Cochrane, 2012, Methods in Enzymology 503, 223-251 and references cited therein).

From these proteins, multimeric and multispecific assemblies can be constructed (Caravella and Lugovskoy, 2010, Current Opinions in Chemical Biology, 14, 520-528; Vanlandschoot et al., 2011, Antiviral Research 92, 389-407; Lofblom et al., 2011, Current Opinion in Biotechnology, 22, 843-848, Boersma et al., 2011, Curr. Opin. Biotechnol., 22, 849-857). It is also possible to fuse these and other peptidic domains to antibodies to create so-called Zybodies (Zyngenia Inc., Gaithersburg, MD).

All of these scaffolds, with different inherent properties, have in common that they can be directed to bind specific epitopes, by using selection technologies well known to practitioners in the field (Binz et al., ibid.).

For example, the different individual domains of HER2 can be individually expressed in insect cells, using a baculovirus expression system, as demonstrated for domain 1 and domain 4 (Frei et al., 2012, Nat Biotechnol., 30, 997-1001). Thereby, it is guaranteed that binders selected will be directed towards the domain of interest. The HER2 domains can then be biotinylated as previously described (Zahnd et al., 2006, J. Biol. Chem. 281(46), 35167-75), and thus be immobilized on streptavidin-coated magnetic beads or on microtiter plates coated with streptavidin or neutravidin (Steiner et al., 2008, J. Mol. Biol. 382, 1211-1227; Zahnd et al., 2007, J. Mol. Biol. 369, 1015-1028.). The HER2 domains so immobilized can then serve as targets for diverse protein libraries in either phage display or ribosome display format. A large variety of different antibody libraries has been published (Mondon P.

et al., 2008, Frontiers in Bioscience. 13, 1117-1129) and the technology of selecting binding antibodies is well known to the practitioners of the field. Phage display is a suitable format for antibody fragments (Fab fragments, scFv fragments or single domain antibodies s) (Hoogenboom, 2005, Nature Biotechnology., 23(9), 1105-1116) and any other scaffold that contain disulfide bonds, but it can also be used for scaffolds not containing disulfide bonds (e.g., Steiner et al., 2008, J. Mol. Biol., 382, 1211-1227) (Rentero et al., 2011, Chimia., 65(11), 843-5, Skerra A., 2007, Current Opinion in Biotechnology., 18(4), 295-304). Similarly, ribosome display can be used for antibody fragments (Hanes et al., 2000, Nat. Biotechnol., 18, 1287-1292) and for other scaffolds (Zahnd et al., 2007, Nat. Methods, 4, 269-279; Zahnd et al., 2007, J. Mol. Biol., 369, 1015-28.). A third powerful technology is yeast display (Pepper et al., 2008, Combinatorial Chemistry & High Throughput Screening, 11(2), 127-134). In this case a library of the binding protein of interest is displayed on the surface of yeast, and the respective domain of HER2 is either directly labeled with a fluorescent dye or its his tag is detected with an anti-histag antibody, which is in turn detected with a secondary antibody. Such methods are well known to the practitioners in the field (Boder et al., 2000, Methods in Enzymology, 328, 430-44).

Another possibility of engineering represents the connection of those binders to create bispecific or higher multivalent binding molecules. Such connection can be achieved genetically by fusions of two or more of these binding molecules or chemically by crosslinking separately expressed molecules, or by adding a dimerization domain include separate dependent claims for each or any combination thereof (see, e.g. Stefan et al., 2011, J. Mol. Biol., 413, 826-843; Boersma et al., 2011, J. Biol. Chem., 286, 41273-41285).

A bispecific anti-HER2 camelidae antibody construct (Bispecific Nanobody) is shown in US20110059090. The document relates to a bispecific molecule that simultaneously targets HER2 at the extracellular domain 2, defined by competition with pertuzumab, and domain 4, defined by competition with trastuzumab. This molecule has been described to exhibit stronger anti-proliferative activity than trastuzumab (Herceptin) in a direct comparison in an in vitro cell culture model using the cell line SkBr3.

Due to the absence of any known HER2-specific ligand, current HER2 targeting strategies aim to block the dimerization of the receptor by binding to the interaction interface. Today's knowledge of HER2 receptor dimerization is mostly based on the crystal structure of the ligand-bound form of the EGFR homodimer, which is broadly accepted as the active mode of all EGF receptor family members (Garret et al., 2002, Cell, 110, 763-773). The two EGFR molecules show a back-to-back interaction. Extending these findings to HER2 and its possible interaction with other members of the EGFR family, one interaction interface is present on domain 2 of the extracellular part of HER2. Pertuzumab binds to domain 2 and is indeed known to block receptor interaction at this interface. Another known interaction is present on domain 4 of the extracellular part of HER2. This interaction interface is presumably blocked by trastuzumab. Yet both antibodies, trastuzumab and pertuzumab, even when simultaneously applied, are not able to block all HER2 interactions to completeness. The interaction of the extracellular part and the kinase domain of HER2 are thought to be linked in such a way as to allow some residual interactions even in the trastuzumab- and pertuzumab-blocked state, which is in accordance with crystal structure data (Lu et al., 2010, Mol. Cell. Biol., 22, 5432-5443). The bispecific ligand mentioned above that binds both epitopes (pertuzumab and trastuzumab) simultaneously (US 2011/0059090) reduces the cell growth in a cell culture model by approx. 50%, in comparison to a reduction of about 40% effected by trastuzumab. This same effect, however, can also be achieved by treating with the mixture of trastuzumab and pertuzumab.

Patent application WO 2014/060365 describes bispecific HER2-targeting agents comprising a first polypeptide ligand that binds to HER2 extracellular domain 1 (D1 epitope) and a second polypeptide ligand that binds to HER2 extracellular domain 4 (D4 epitope), wherein the first and the second polypeptide ligand are separated by linker. The most active ones of these biparatopic binding agents bind predominantly to two separate HER2 molecules in an intermolecular binding mode. Thereby these biparatopic bivalent binding agents crosslink HER2ECD1 of one HER2 and HER2_ECD4 of the other HER2 molecule via these paratopes and a preferentially short peptide linker, which favors inter- over intramolecular binding. The bivalent binding mode of the biparatopic binding agents will induce a polymerization of HER2 receptors at the cell surface, which are consequently not able to form productive HER2/HER3 or HER2/EGFR heterodimers or HER2/HER2 homodimers (Tamaskovic et al., 2016, Jost et al., 2013). This consequently leads to an inhibition of HER2 and HER3 phosphorylation in HER2-overexpressing cancer cells, which subsequently leads to cessation of proliferative and anti-apoptotic signaling from HER2/HER3 receptor and finally induction of cell death by apoptosis. However, these biparatopic binding agents do not affect the total HER2 receptor expression levels (Tamaskovic et al., 2016). The total HER2 receptor expression remains comparably high, which may cause an incomplete inactivation of HER2 receptors. Furthermore, these constructs do not show the desired pharmacokinetic properties, which are required for systemic applications. Desired functions such as long serum half-life in the blood stream or mechanisms of protein recycling via binding to the FcRn receptor are not implemented in these constructs. Furthermore, these biparatopic constructs do not harness the antibody effector functions such as complement-dependent cytotoxicity (CDC) or antibody-dependent cell-mediated cytotoxicity (ADCC). These effector functions may be beneficial to increase further the anti-tumor activity of the biparatopic binding agents in vivo. Finally, these biparatopic binding agents are potentially prone to induce an immune response, because they have not been further engineered to avoid T-cell epitopes. This may lead to significant reduction of tolerability and serum level at repeated dosage in immunocompetent patients.

Furthermore, a tetravalent biparatopic HER2-targeting antibody-drug conjugate comprising a fusion of Trastuzumab and 39S antibody variable sequences binding to the D4 and D2 epitopes of HER2 has been described (Li et al., 2016, Cancer Cell, 29, 117-129). This conjugate was commercialized under the name MED14276 by Medimmune and was tested in a clinical phase 1/2 trial (NCT02576548). However, this biparatopic IgG fusion construct in its unarmed form, i.e., without the fusion of the Tubulysine toxic payload, is not sufficient to induce inhibition of cancer cell proliferation. On the contrary, the unconjugated version of this IgG fusion molecule induces activation of cancer cell proliferation at high concentrations in HER2-overexpressing cancer cell models. This may be caused by the binding to HER2_ECD2 and HER2_ECD4 in a manner that increases the formation of signaling-competent HER2 homo- and heterodimers. On the other hand, the unconjugated IgG fusion protein version of this tetravalent biparatopic HER2-targeting antibody can induce downregulation of HER2 receptor expression (Li et al., 2016, Cancer Cell, 29, 117-129). In summary, the tetravalent scFv_4D5-IgG_39S fusion protein downregulates HER2 expression, yet it shows no inhibition of HER2 signaling and instead leads to activation of cancer cell proliferation in specific HER2-overexpressing models. This also shows that downregulation of HER2 and cancer cell growth inhibition are not simply linked. Clearly, inhibition of HER2-dependent signaling pathways of a HER2-targeting agent would be essential for clinical applications. For example, trastuzumab (Herceptin), does block signaling of HER3 and does show significant reduction of cell proliferation in HER2-overexpressing cancers that do not have a PI3K-pathway activating mutation.

Based on the above-mentioned state of the art, the objective of the present invention is to provide means and methods to provide a HER2-targeting agent which is improved in view of the above-stated disadvantages of the prior art, in particular to provide a HER2-targeting agent displaying improved HER2 signalling inhibition, downregulation of expression, polymerization and clustering, inhibition of receptor diffusion, degradation, and/or inhibition of recycling, in the absence of additional small molecule toxins bound to the HER2 targeting agent. This objective is attained by the subject-matter of the independent claims of the present specification.

### SUMMARY OF THE INVENTION

The invention relates to a tetrameric polypeptide comprising or consisting of
a first polypeptide chain comprising in N to C orientation a first VL antigen binding domain and a first CL constant domain,
a second polypeptide chain comprising in N to C orientation a first VH antigen binding domain, a first CH1 constant domain, a first CH2 constant domain and a first CH3 constant domain,
a first ligand that specifically binds to a HER2 D4 epitope, wherein the first ligand is comprised in the first polypeptide chain and linked to the N-terminus of the first VL antigen binding domain by a first interdomain amino acid linker, or the first ligand is comprised in the second polypeptide chain and linked to the N-terminus of the first VH antigen binding domain by a first interdomain amino acid linker,
wherein the first VL antigen binding domain of the first polypeptide chain and the first VH antigen binding domain of the second polypeptide chain together constitute a second ligand, particularly a Fab domain, that specifically binds to a HER2 D1 epitope,
a third polypeptide chain comprising in N to C orientation a second VL antigen binding domain and a second CL constant domain,
a fourth polypeptide chain comprising in N to C orientation a second VH antigen binding domain, a second CH1 constant domain, a second CH2 constant domain and a second CH3 constant domain,
a third ligand that specifically binds to a HER2 D4 epitope, wherein the third ligand is comprised in the third polypeptide chain and linked to the N-terminus of the second VL antigen binding domain by a second interdomain amino acid linker, or the third ligand is comprised in the fourth polypeptide chain and linked to the N-terminus of the second VH antigen binding domain by a second interdomain amino acid linker,
wherein the second VL antigen binding domain of the third polypeptide chain and the second VH antigen binding domain of the fourth polypeptide chain together constitute a fourth ligand, particularly an Fab domain, that specifically binds to a HER2 D1 epitope.

The tetrameric polypeptide according to the invention thus comprises two times two different HER2-binding paratopes, in other words, is tetravalent. The polypeptide is biparatopic, because it contains binding sites to two distinct HER2 epitopes, namely D1 and D4 on a single molecule.

Surprisingly, the polypeptide displays superior HER2 inactivation compared to conventional antibodies and divalent biparatopic polypeptides (comprised of a total of two binding paratopes) and has additional effects on cell growth and proliferation, apoptosis and other forms of cell death, HER2 internalization and HER2 recycling inhibition, HER2 expression downregulation and HER2 degradation, HER2 crosslinking, inhibition of HER2 dimerization, and decrease of HER2 receptor surface mobility. Furthermore, the increased molecular size excludes renal filtration, and FcRn-mediated recycling will increase the pharmacokinetic properties. Finally, the presence of the Fc part also allows antibody-dependent cell-mediated cytotoxicity (ADCC) and complement-dependent cytotoxicity (CDC) to occur. Lastly, all sequences are those of human antibodies, and thus the construct consists of non-immunogenic sequences. In summary, the tetrameric polypeptides according to the present invention are promising candidates for systemic therapy of HER2-expressing cancer.

In another embodiment, the present invention relates to a pharmaceutical composition comprising at least one of the compounds of the present invention or a pharmaceutically acceptable salt thereof and at least one pharmaceutically acceptable carrier, diluent or excipient.

The invention further relates to the tetrameric polypeptide for use in a method for the prevention or treatment of a malignant neoplastic disease, an isolated nucleic acid encoding the polypeptide, a host cell for producing the polypeptide and a method for obtaining the polypeptide.

### DETAILED DESCRIPTION OF THE INVENTION

Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term “a subject comprises an object” in the context of the present specification includes discrete embodiments where the subject consists of the object, in other words where the term comprise is synonymous with “consist of”. In other discrete embodiments, the object is one of several different comprised in the object.

The term ligand in the context of the present specification relates to a region of a polypeptide binding to a target, particularly HER2.

The term interdomain amino acid linker in the context of the present specification relates to a polypeptide linker covalently connecting the C-terminus of a first polypeptide domain to the N-terminus of a second polypeptide domain.

The term epitope in the context of the present specification relates to a region of an antigen molecule to which an antibody binds.

The term Fab domain in the context of the present specification relates to an antibody molecule comprising a first chain consisting of a VL domain C-terminally (covalently) linked to a CL domain and a second chain consisting of a VH domain C- terminally (covalently) linked to a CH1 domain, wherein the CL domain and the CH1 domain are linked by a disulfide bond.

The term VL antigen binding domain in the context of the present specification relates to the variable domain of the light chain of an antibody, particularly of an immunoglobulin G light chain.

The term VH antigen binding domain in the context of the present specification relates to the variable domain of the heavy chain of an antibody, particularly of an immunoglobulin G light chain.

The term CL constant domain in the context of the present specification relates to the constant domain of the light chain of an antibody, particularly of an immunoglobulin G heavy chain.

The term CH1 constant domain in the context of the present specification relates to the first constant domain of the heavy chain of an antibody, particularly of an immunoglobulin G heavy chain.

The term CH2 constant domain in the context of the present specification relates to the second constant domain of the heavy chain of an antibody, particularly of an immunoglobulin G heavy chain.

The term CH3 constant domain in the context of the present specification relates to the third constant domain of the heavy chain of an antibody, particularly of an immunoglobulin G heavy chain.

The term single-chain variable fragment in the context of the present specification relates to a VH antigen binding domain (also termed scFv heavy chain) covalently linked to a VL antigen binding domain (also termed scFv light chain) by a polypeptide linker (scFv linker chain).

In the present specification, the term positive, when used in the context of expression of a marker, refers to expression of an antigen assayed by a fluorescently labelled antibody, wherein the label's fluorescence on the structure (for example, a cell) referred to as “positive” is at least 30% higher 30%), particularly≥50% or ≥80%, in median fluorescence intensity in comparison to staining with an isotype-matched fluorescently labelled antibody which does not specifically bind to the same target. Such expression of a marker is indicated by a superscript “plus” (+), following the name of the marker, e.g. CD4⁺.

In the present specification, the term negative, when used in the context of expression of a marker, refers to expression of an antigen assayed by a fluorescently labelled antibody, wherein the median fluorescence intensity is less than 30% higher, particularly less than 15% higher, than the median fluorescence intensity of an isotype-matched antibody which does not specifically bind the same target. Such expression of a marker is indicated by a superscript minus (−), following the name of the marker, e.g. CD127⁻.

High expression of a marker, for example high expression of CD25, refers to the expression level of such marker in a clearly distinguishable cell population that is detected by FACS showing the highest fluorescence intensity per cell compared to the other populations characterized by a lower fluorescence intensity per cell. A high expression is indicated by superscript “high” or “hi” following the name of the marker, e.g. CD25^{high}. The term “is expressed highly” refers to the same feature.

Low expression of a marker, for example low expression of CD25, refers to the expression level of such marker in a clearly distinguishable cell population that is detected by FACS showing the lowest fluorescence intensity per cell compared to the other populations characterized by higher fluorescence intensity per cell. A low expression is indicated by superscript “low” or “lo” following the name of the marker, e.g. CD25^{low}. The term “is expressed lowly” refers to the same feature.

The expression of a marker may be assayed via techniques such as fluorescence microscopy, flow cytometry, ELISPOT, ELISA or multiplex analyses.

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). J is leucine or isoleucine.

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or alternatively gene product refer to the processes—and products thereof—of nucleic acids (RNA) or amino acids (e.g., peptide or polypeptide) being generated when a gene is transcribed and translated.

As used herein, expression refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell. Expression may be assayed both on the level of transcription and translation, in other words mRNA and/or protein product.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a particular embodiment, the length of a reference sequence aligned for comparison purposes is at least 30%, particularly at least 40%, more particularly at least 50%, even more particularly at least 60%, and even more particularly at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. In the case of circularly related proteins, the sequence of one of the partners needs to be appropriately split and aligned in two sections to achieve optimal alignment of the functionally equivalent residues necessary to calculate the percent identity.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, 1981, Adv. Appl. Math., 2, 482, by the global alignment algorithm of Needleman and Wunsch, 1970, J. Mol. Biol., 48, 443, by the search for similarity method of Pearson and Lipman, 1988, Proc. Nat. Acad. Sci., 85, 2444 or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., 1990, J. Mol. Biol., 215, 403-410) using the above identified default parameters for protein and nucleic acid comparison, respectively.

In the context of the present specification, the term antibody refers to whole antibodies including but not limited to immunoglobulin type G (IgG), type A (IgA), type D (IgD), type E (IgE) or type M (IgM), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

In the context of the present specification, the term humanized antibody refers to an antibody originally produced by immune cells of a non-human species, the protein sequences of which have been modified to increase their similarity to antibody variants produced naturally in humans. The term humanized antibody as used herein includes antibodies in which CDR sequences derived from the germline of another mammalian species, such as a mouse, have been grafted onto human framework sequences. Additional framework region modifications may be made within the human framework sequences as well as within the CDR sequences derived from the germline of another mammalian species.

The term antibody-like molecule in the context of the present specification refers to a molecule capable of specific binding to another molecule or target with high affinity/a Kd≤5 10E-8 mol/l. An antibody-like molecule binds to its target similarly to the specific binding of an antibody. The term antibody-like molecule encompasses a repeat protein, such as a designed ankyrin repeat protein (Molecular Partners, Zurich), an engineered antibody mimetic proteins exhibiting highly specific and high-affinity target protein binding (see US 2012/142611, US 2016/250341, US 2016/075767 and US 2015/368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins.

The term antibody-like molecule further encompasses a specifically binding polypeptide derived from
a protein A domain,
fibronectin domain FN3,
consensus fibronectin domains,
a lipocalins (see Skerra, 2000, Biochim. Biophys. Acta, 1482(1-2), 337-50),
a polypeptide derived from a Zinc finger protein (see Kwan et al., 2003, Structure, 11(7), 803-813),
Src homology domain 2 (SH2) or Src homology domain 3 (SH3),
a PDZ domain,
gamma-crystallin,
ubiquitin,
a cysteine knot polypeptide or a knottin,
cystatin,
Sac7d,
a triple helix coiled coil (also known as alphabodies),
a Kunitz domain or a Kunitz-type protease inhibitor and
a carbohydrate binding module 32-2.

The term protein A domains derived polypeptide refers to a molecule that is a derivative of protein A and is capable of specifically binding the Fc region and the Fab region of immunoglobulins.

The term armadillo repeat protein refers to a polypeptide comprising at least one armadillo repeat, wherein an armadillo repeat is characterized by a pair of alpha helices that form a hairpin structure.

The term humanized camelid antibody in the context of the present specification refers to an antibody consisting of only the heavy chain or the variable domain of the heavy chain (VHH domain) and whose amino acid sequence has been modified to increase their similarity to antibodies naturally produced in humans and, thus show a reduced immunogenicity when administered to a human being. A general strategy to humanize camelid antibodies is shown in Vincke et al., 2009, J Biol Chem., 284(5), 3273-3284, and US 2011/165621.

In the context of the present specification, the term fragment crystallizable (Fc) region is used in its meaning known in the art of cell biology and immunology; it refers to a fraction of an antibody comprising two identical heavy chain fragments comprised of a C_{H}2 and a C_{H}3 domain, covalently linked by disulfide bonds.

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷ mol/L when binding to its target, but a dissociation constant at least three orders of magnitude higher in its interaction with a molecule having a globally similar chemical composition as the target, but a different three-dimensional structure.

In the context of the present specification, the term dissociation constant (K_{D}) is used in its meaning known in the art of chemistry and physics; it refers to an equilibrium constant that measures the propensity of a complex composed of [mostly two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ Ab ] * [ Ag ]}{[ AbAg ]}$$

[Ab]: concentration of antibody; [Ag]: concentration of antigen; [AbAg]: concentration of antibodyantigen complex

In the context of the present specification, the terms off-rate (Koff;[1/sec]) and on-rate (Kon; [1/sec*M]) are used in their meaning known in the art of chemistry and physics; they refer to a rate constant that measures the dissociation (Koff) or association (Kon) of 5 an antibody with its target antigen. Koff and Kon can be experimentally determined using methods well established in the art. A method for determining the Koff and Kon of an antibody employs surface plasmon resonance. This is the principle behind biosensor systems such as the Biacore® or the ProteOn® system. They can also be used to determine the dissociation constant KD by using the following formula:

$${K}_{D} = \frac{[ {K}_{off} ]}{[ {K}_{on} ]}$$

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

In the context of the present specification, the term dimer refers to a unit consisting of two subunits.

In the context of the present specification, the term homodimer refers to a dimer comprised of two subunits that are either identical or are highly similar members of the same class of subunits.

In the context of the present specification, the term amino acid linker refers to a polypeptide of variable length that is used to connect two polypeptides in order to generate a single chain polypeptide. Exemplary embodiments of linkers useful for practicing the invention specified herein are oligopeptide chains consisting of 1, 2, 3, 4, 5, 10, 20, 30, 40 or 50 amino acids. A non-limiting example of an amino acid linker is the polypeptide GGGGSGGGGS (SEQ ID NO 83).

A first aspect of the invention relates to a tetrameric polypeptide

According to a first alternative of the first aspect, the tetrameric polypeptide comprises or consists of
a first polypeptide chain comprising in N to C orientation a first VL antigen binding domain and a first CL constant domain,
a second polypeptide chain comprising in N to C orientation a first VH antigen binding domain, a first CH1 constant domain, a first CH2 constant domain and a first CH3 constant domain,
a first ligand that specifically binds to a HER2 D4 epitope, wherein the first ligand is comprised in the first polypeptide chain and linked to the N-terminus of the first VL antigen binding domain by a first interdomain amino acid linker, or the first ligand is comprised in the second polypeptide chain and linked to the N-terminus of the first VH antigen binding domain by a first interdomain amino acid linker,
wherein the first VL antigen binding domain of the first polypeptide chain and the first VH antigen binding domain of the second polypeptide chain together constitute a second ligand, particularly a Fab domain, that specifically binds to a HER2 D1 epitope,
a third polypeptide chain comprising in N to C orientation a second VL antigen binding domain and a second CL constant domain,
a fourth polypeptide chain comprising in N to C orientation a second VH antigen binding domain, a second CH1 constant domain, a second CH2 constant domain and a second CH3 constant domain,
a third ligand that specifically binds to a HER2 D4 epitope, wherein the third ligand is comprised in the third polypeptide chain and linked to the N-terminus of the second VL antigen binding domain by a second interdomain amino acid linker, or the third ligand is comprised in the fourth polypeptide chain and linked to the N-terminus of the second VH antigen binding domain by a second interdomain amino acid linker,
wherein the second VL antigen binding domain of the third polypeptide chain and the second VH antigen binding domain of the fourth polypeptide chain together constitute a fourth ligand, particularly an Fab domain, that specifically binds to a HER2 D1 epitope.

According to a second alternative of the first aspect, the tetrameric polypeptide comprises or consists of
a first polypeptide chain comprising in N to C orientation a first VL antigen binding domain and a first CL constant domain,
a second polypeptide chain comprising in N to C orientation a first VH antigen binding domain and a first CH1 constant domain, wherein particularly the second polypeptide chain further comprises a first CH2 constant domain, wherein more particularly the first CH2 constant domain is truncated at its C-terminus,
a first ligand that specifically binds to a HER2 D4 epitope, wherein the first ligand is comprised in the first polypeptide chain and linked to the N-terminus of the first VL antigen binding domain by a first interdomain amino acid linker, or the first ligand is comprised in the second polypeptide chain and linked to the N-terminus of the first VH antigen binding domain by a first interdomain amino acid linker,
wherein the first VL antigen binding domain of the first polypeptide chain and the first VH antigen binding domain of the second polypeptide chain together constitute a second ligand, particularly a Fab domain, that specifically binds to a HER2 D1 epitope,
a third polypeptide chain comprising in N to C orientation a second VL antigen binding domain and a second CL constant domain,
a fourth polypeptide chain comprising in N to C orientation a second VH antigen binding domain and a second CH1 constant domain, wherein particularly the fourth polypeptide chain further comprises a second CH2 constant domain, wherein more particularly the second CH2 constant domain is truncated at its C-terminus,
a third ligand that specifically binds to a HER2 D4 epitope, wherein the third ligand is comprised in the third polypeptide chain and linked to the N-terminus of the second VL antigen binding domain by a second interdomain amino acid linker, or the third ligand is comprised in the fourth polypeptide chain and linked to the N-terminus of the second VH antigen binding domain by a second interdomain amino acid linker,
wherein the second VL antigen binding domain of the third polypeptide chain and the second VH antigen binding domain of the fourth polypeptide chain together constitute a fourth ligand, particularly an Fab domain, that specifically binds to a HER2 D1 epitope.

All embodiments below may be combined with both the first alternative and the second alternative of the first aspect of the invention.

The VL antigen binding domain and the CL constant domain of the first and third polypeptide chain are domains of an immunoglobulin G light chain, and the VH antigen binding domain and the CH1, CH2 and CH3 constant domains of the second and fourth polypeptide are domains of an immunoglobulin G heavy chain. In other words, the first and the third polypeptide chains each comprise an immunoglobulin G light chain, and the second and the fourth polypeptide chains each comprise an immunoglobulin G heavy chain. The immunoglobulin light and heavy chains of the tetrameric polypeptide according to the invention form the second and fourth ligands specifically binding to HER2 D1 epitope.

In addition, polypeptides comprising a first and third ligand which specifically binds to HER2 D4 epitope are fused to the N-terminus of the immunoglobulin G heavy chains or immunoglobulin G light chains by an interdomain amino acid linker resulting in a tetrameric polypeptide having four HER2 binding sites, two of which bind to the D4 epitope and two of which bind to the D1 epitope.

Surprisingly, the tetrameric polypeptide according to the invention displays superior HER2 inactivation compared to conventional antibodies and other antibody-like molecules, such as divalent biparatopic polypeptides (having a total of two binding regions) and has additional effects on cell growth, apoptosis, HER2 internalization and HER2 degradation. Therefore, the tetrameric polypeptides according to the present invention are promising candidates for therapy of HER2-expressing cancer.

Without wishing to be bound be theory, it is believed that the CH1, CH2, CH3 and CL domains contribute, particularly the CH2 and CH3 domains, to the additional effects of the tetrameric polypeptides of the invention, particularly inhibition of cell growth and proliferation, induction of apoptosis and other forms of cell death, HER2 internalization, HER2 recycling inhibition and HER2 degradation, HER2 crosslinking, reduction of HER2 surface mobility, HER2 expression downregulation, inhibition of HER2 dimerization and signalling by positioning the variable domains at a particular angle and distance.

In certain embodiments, the first polypeptide chain and the third polypeptide chain comprise a sequence identity with each other of 70% or more, particularly 80% or more, more particularly 90% or more, even more particularly 95% or more, wherein most particularly the first polypeptide chain and the third polypeptide chain are identical.

In certain embodiments, the first polypeptide chain comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the third polypeptide chain.

In certain embodiments, the second polypeptide chain and the fourth polypeptide chain comprise a sequence identity with each other of 70% or more, particularly 80% or more, more particularly 90% or more, even more particularly 95% or more, wherein most particularly the second polypeptide chain and the fourth polypeptide chain are identical.

In certain embodiments, the second polypeptide chain comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the fourth polypeptide chain.

In certain embodiments, the immunoglobulin light chain of the first polypeptide chain and the immunoglobulin light chain of the third polypeptide chain comprise a sequence identity with each other of 70% or more, particularly 80% or more, more particularly 90% or more, even more particularly 95% or more, wherein most particularly the immunoglobulin light chain of the first polypeptide chain and the immunoglobulin light chain of the third polypeptide chain are identical.

In certain embodiments, the immunoglobulin light chain of the first polypeptide chain comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the immunoglobulin light chain of the third polypeptide chain.

In certain embodiments, the immunoglobulin heavy chain of the second polypeptide chain and the immunoglobulin heavy chain of the fourth polypeptide chain comprise a sequence identity with each other of 70% or more, particularly 80% or more, more particularly 90% or more, even more particularly 95% or more, wherein most particularly the immunoglobulin heavy chain of the second polypeptide chain and the immunoglobulin heavy chain of the fourth polypeptide chain are identical.

In certain embodiments, the immunoglobulin heavy chain of the second polypeptide chain comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the immunoglobulin heavy chain of the fourth polypeptide chain.

In certain embodiments, the first ligand and the third ligand comprise a sequence identity with each other of 70% or more, particularly 80% or more, more particularly 90% or more, even more particularly 95% or more, wherein most particularly the first ligand and the third ligand are identical.

In certain embodiments, the first ligand comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the third ligand.

In other words, the first ligand is substantially the same as the third ligand.

In certain embodiments, the second ligand and the fourth ligand comprise a sequence identity with each other of 70% or more, particularly 80% or more, more particularly 90% or more, even more particularly 95% or more, wherein most particularly the first ligand and the third ligand are identical.

In certain embodiments, the second ligand comprises a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the fourth ligand.

In other words, the second ligand is substantially the same as the fourth ligand.

In certain embodiments, the first CH2 constant domain and the first CH3 constant domain of the second polypeptide chain interact with the second CH2 constant domain and the second CH3 constant domain of the fourth polypeptide chain, such that a tetrameric polypeptide is formed. In particular, the CH2 and CH3 constant domains of the second and fourth polypeptide chain dimerize. In particular, combined with the interaction between the first and the second polypeptide chains and the third and the fourth polypeptide chains, particularly by means of the dimerization between the respective VL and VH antigen binding domains and the CL and CH1 constant domains, this results in tetramer formation of the first, second, third and fourth polypeptide. Therefore, in respect of multimer formation, the tetrameric polypeptide of the present invention is similar to an antibody.

In certain embodiments, the second polypeptide chain comprises a first hinge region between the first CH1 constant domain and the first CH2 constant domain, and the fourth polypeptide chain comprises a second hinge region between the second CH1 constant domain and the second CH2 constant domain, wherein the first hinge region and the second hinge region mediate complex formation between the second polypeptide chain and the fourth polypeptide chain, particularly by at least one disulphide bond, more particularly by a first disulphide bond and a second disulphide bond, such that a tetrameric polypeptide is formed. Complex formation between the CH1 and CH2 constant domains may thus occur by the hinge region, i.e. by disulphide bond formation between cysteine residues, just as in antibodies.

In certain embodiments, the CH2 constant domain and/or the CH3 constant domain of the second polypeptide chain is truncated at its C-terminus.

In certain embodiments, the CH2 constant domain and/or the CH3 constant domain of the fourth polypeptide chain is truncated at its C-terminus.

In certain embodiments, the first ligand comprises or consists of a single-chain variable fragment polypeptide chain comprising an scFv heavy chain, an scFv linker chain, and an scFv light chain. In certain embodiments, the scFv heavy chain is the VH domain of 4D5 (particularly SEQ ID No. 80), and wherein the scFv light chain is the VL domain of 4D5 (particularly SEQ ID No. 81).

In certain embodiments, the single-chain variable fragment polypeptide chain comprises in N to C orientation an scFv heavy chain, an scFv linker chain, and an scFv light chain.

In certain embodiments, the single-chain variable fragment polypeptide chain comprises in N to C orientation an scFv light chain, an scFv linker chain, and an scFv heavy chain.

In other words, the the scFv heavy chain and the scFv light chain may be provided in any orientation on the single-chain variable fragment polypeptide chain.

In certain embodiments, the third ligand comprises or consists of a single-chain variable fragment polypeptide chain comprising in N to C orientation an scFv heavy chain, an scFv linker chain, and an scFv light chain. In certain embodiments, the scFv heavy chain is the VH domain of 4D5 (particularly SEQ ID No. 80), and wherein the scFv light chain is the VL domain of 4D5 (particularly SEQ ID No. 81).

In certain embodiments,
the scFv heavy chain of the first ligand comprises or consists of a peptide sequence having a sequence identity of 70% or more, particularly 80% or more, more particularly 90% or more, even more particularly 95% or more, with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, wherein most particularly the scFv heavy chain of the first ligand comprises or consists of a peptide sequence identical to a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the first ligand comprises or consists of a peptide sequence having a sequence identity of 70% or more, particularly 80% or more, more particularly 90% or more, even more particularly 95% or more, with a peptide sequence selected from SEQ ID No. 14, SEQ ID No. 24, SEQ ID No. 25, SEQ ID No. 26, SEQ ID No. 43 and SEQ ID No. 81, wherein most particularly the scFv light chain of the first ligand comprises or consists of a peptide sequence identical to a peptide sequence selected from SEQ ID No. 14, SEQ ID No. 24, SEQ ID No. 25, SEQ ID No. 26, SEQ ID No. 43 and SEQ ID No. 81.

In certain embodiments,
the scFv heavy chain of the first ligand comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the first ligand comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 14, SEQ ID No. 24, SEQ ID No. 25, SEQ ID No. 26, SEQ ID No. 43 and SEQ ID No. 81.

In certain embodiments,
the scFv heavy chain of the third ligand comprises or consists of a peptide sequence having a sequence identity of 70% or more, particularly 80% or more, more particularly 90% or more, even more particularly 95% or more, with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, wherein most particularly the scFv heavy chain of the third ligand comprises or consists of a peptide sequence identical to a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the third ligand comprises or consists of a peptide sequence having a sequence identity of 70% or more, particularly 80% or more, more particularly 90% or more, even more particularly 95% or more, with a peptide sequence selected from SEQ ID No. 14, SEQ ID No. 24, SEQ ID No. 25, SEQ ID No. 26, SEQ ID No. 43 and SEQ ID No. 81, wherein most particularly the scFv light chain of the third ligand comprises or consists of a peptide sequence identical to a peptide sequence selected from SEQ ID No. 14, SEQ ID No. 24, SEQ ID No. 25, SEQ ID No. 26, SEQ ID No. 43 and SEQ ID No. 81.

In certain embodiments,
the scFv heavy chain of the third ligand comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the third ligand comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 14, SEQ ID No. 24, SEQ ID No. 25, SEQ ID No. 26, SEQ ID No. 43 and SEQ ID No. 81.

In certain embodiments,
the scFv heavy chain of the first ligand and the third ligand each comprises or consists of a peptide sequence having a sequence identity of 70% or more, particularly 80% or more, more particularly 90% or more, even more particularly 95% or more, with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, wherein most particularly the scFv heavy chain of the first ligand and the third ligand each comprises or consists of a peptide sequence identical to a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the first ligand and the third ligand each comprises or consists of a peptide sequence having a sequence identity of 70% or more, particularly 80% or more, more particularly 90% or more, even more particularly 95% or more, with a peptide sequence selected from SEQ ID No. 14, SEQ ID No. 24, SEQ ID No. 25, SEQ ID No. 26, SEQ ID No. 43 and SEQ ID No. 81, wherein most particularly the scFv light chain of the first ligand and the third ligand each comprises or consists of a peptide sequence identical to a peptide sequence selected from SEQ ID No. 14, SEQ ID No. 24, SEQ ID No. 25, SEQ ID No. 26, SEQ ID No. 43 and SEQ ID No. 81.

In certain embodiments,
the scFv heavy chain of the first ligand and the third ligand each comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and
the scFv light chain of the first ligand and the third ligand each comprises or consists of a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 14, SEQ ID No. 24, SEQ ID No. 25, SEQ ID No. 26, SEQ ID No. 43 and SEQ ID No. 81.

That is the scFv light and heavy chains may completely consist of the above-defined peptide sequence or the scFv light and heavy chains may comprise the above-defined peptide sequence, wherein the scFv light and heavy chains may contain additional peptide sequences.

In certain embodiments, the scFv light chain of the first ligand comprises or consists of the VL antigen binding domain of antibody 4D5 and the scFv heavy chain of the first ligand comprises or consists of the VH antigen binding domain of antibody 4D5.

In certain embodiments, the scFv light chain of the third ligand comprises or consists of the VL antigen binding domain of antibody 4D5 and the scFv heavy chain of the third ligand comprises or consists of the VH antigen binding domain of antibody 4D5.

In the context of the present specification, the term 4D5 refers to the humanized monoclonal antibody trastuzumab, also known as Herceptin, and also referred to herein as “TZB” which is directed against the membrane-proximal domain IV of HER2 (Cho et al., 2003).

In certain embodiments, the scFv linker chain comprises a peptide sequence having a sequence identity of 70% or more, particularly 80% or more, more particularly 90% or more, even more particularly 95% or more, with SEQ ID No. 16, wherein most particularly the scFv linker chain comprises a peptide sequence identical to SEQ ID No. 16.

In certain embodiments, the scFv linker chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 16.

In certain embodiments,
the first polypeptide chain comprises a peptide sequence having a sequence identity of 70% or more, particularly 80% or more, more particularly 90% or more, even more particularly 95% or more, with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, wherein most particularly the first polypeptide chain comprises a peptide sequence identical to a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, and
the second polypeptide chain comprises a peptide sequence having a sequence identity of 70% or more, particularly 80% or more, more particularly 90% or more, even more particularly 95% or more, with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77, wherein most particularly the second polypeptide chain comprises a peptide sequence identical to a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

In certain embodiments,
the first polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76,
the second polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

In certain embodiments,
the third polypeptide chain comprises a peptide sequence having a sequence identity of 70% or more, particularly 80% or more, more particularly 90% or more, even more particularly 95% or more, with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, wherein most particularly the third polypeptide chain comprises a peptide sequence identical to a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, and
the fourth polypeptide chain comprises a peptide sequence having a sequence identity of 70% or more, particularly 80% or more, more particularly 90% or more, even more particularly 95% or more, with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77, wherein most particularly the fourth polypeptide chain comprises a peptide sequence identical to a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

In certain embodiments,
the third polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76,
the fourth polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

In certain embodiments,
the first polypeptide chain and the third polypeptide chain each comprises a peptide sequence having a sequence identity of 70% or more, particularly 80% or more, more particularly 90% or more, even more particularly 95% or more, with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, wherein most particularly the first polypeptide chain and the third polypeptide chain each comprises a peptide sequence identical to a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, and
the second polypeptide chain and the fourth polypeptide chain each comprises a peptide sequence having a sequence identity of 70% or more, particularly 80% or more, more particularly 90% or more, even more particularly 95% or more, with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77, wherein most particularly the second polypeptide chain and the fourth polypeptide chain each comprises a peptide sequence identical to a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

In certain embodiments,
the first polypeptide chain and the third polypeptide chain each comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76,
the second polypeptide and the fourth polypeptide chain each comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

Therefore, the VL and VH antigen binding domain of the first, second, third and fourth polypeptide chains may be substantially identical to the VL and VH antigen binding domains of the scFv fragment termed A21 (Hu S. et al., 2008, Proteins, 70, 938-949) which specifically binds to domain I of HER2.

In certain embodiments,
the first polypeptide chain comprises a peptide sequence having a sequence identity of 70% or more, particularly 80% or more, more particularly 90% or more, even more particularly 95% or more, with SEQ ID No. 36, SEQ ID No. 37, SEQ ID No. 38 and SEQ ID No. 78, wherein most particularly the first polypeptide chain comprises a peptide sequence identical to SEQ ID No. 36, SEQ ID No. 37, SEQ ID No. 38 and SEQ ID No. 78, and
the second polypeptide chain comprises a peptide sequence having a sequence identity of 70% or more, particularly 80% or more, more particularly 90% or more, even more particularly 95% or more, with SEQ ID No. 33, SEQ ID No. 34, SEQ ID No. 35 and SEQ ID No. 79, wherein most particularly the second polypeptide chain comprises a peptide sequence identical to SEQ ID No. 33, SEQ ID No. 34, SEQ ID No. 35 and SEQ ID No. 79.

In certain embodiments,
the first polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 36, SEQ ID No. 37, SEQ ID No. 38 and SEQ ID No. 78, and
the second polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 33, SEQ ID No. 34, SEQ ID No. 35 and SEQ ID No. 79.

In certain embodiments,
the third polypeptide chain comprises a peptide sequence having a sequence identity of 70% or more, particularly 80% or more, more particularly 90% or more, even more particularly 95% or more, with SEQ ID No. 36, SEQ ID No. 37, SEQ ID No. 38 and SEQ ID No. 78, wherein most particularly the third polypeptide chain comprises a peptide sequence identical to SEQ ID No. 36, SEQ ID No. 37, SEQ ID No. 38 and SEQ ID No. 78, and
the fourth polypeptide chain comprises a peptide sequence having a sequence identity of 70% or more, particularly 80% or more, more particularly 90% or more, even more particularly 95% or more, with SEQ ID No. 33, SEQ ID No. 34, SEQ ID No. 35 and SEQ ID No. 79, wherein most particularly the fourth polypeptide chain comprises a peptide sequence identical to SEQ ID No. 33, SEQ ID No. 34, SEQ ID No. 35 and SEQ ID No. 79.

In certain embodiments,
the third polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 36, SEQ ID No. 37, SEQ ID No. 38 and SEQ ID No. 78, and
the fourth polypeptide chain comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 33, SEQ ID No. 34, SEQ ID No. 35 and SEQ ID No. 79.

In certain embodiments,
the first polypeptide chain and the third polypeptide chain each comprises a peptide sequence having a sequence identity of 70% or more, particularly 80% or more, more particularly 90% or more, even more particularly 95% or more, with SEQ ID No. 36, SEQ ID No. 37, SEQ ID No. 38 and SEQ ID No. 78, wherein most particularly the first polypeptide chain and the third polypeptide chain each comprises a peptide sequence identical to SEQ ID No. 36, SEQ ID No. 37, SEQ ID No. 38 and SEQ ID No. 78, and
the second polypeptide chain and the fourth polypeptide chain each comprises a peptide sequence having a sequence identity of 70% or more, particularly 80% or more, more particularly 90% or more, even more particularly 95% or more, with SEQ ID No. 33, SEQ ID No. 34, SEQ ID No. 35 and SEQ ID No. 79, wherein most particularly the second polypeptide chain and the fourth polypeptide chain each comprises a peptide sequence identical to SEQ ID No. 33, SEQ ID No. 34, SEQ ID No. 35 and SEQ ID No. 79.

In certain embodiments,
the first polypeptide chain and the third polypeptide chain each comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 36, SEQ ID No. 37, SEQ ID No. 38 and SEQ ID No. 78, and
the second polypeptide chain and the fourth polypeptide chain each comprises a peptide sequence having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 33, SEQ ID No. 34, SEQ ID No. 35 and SEQ ID No. 79.

Therefore, the immunoglobulin light and heavy chains of the first, second, third and fourth polypeptide chains may comprise IgG domains (VL, CL, VH, CH1, CH2 and/or CH3) substantially identical to the corresponding domains of the ErbB2 antibody termed 7C2 (U.S. Pat. No. 7,371,376) which specifically binds to domain I of HER2.

In certain particular embodiments, the tetrameric polypeptide according to the invention is essentially an immunoglobulin G type antibody (particularly a human or humanized monoclonal IgG antibody) having two identical heavy chains and two identical light chains, wherein the antigen specific variable heavy and light chains together form a ligand (the second and fourth ligand) specifically reactive to the D1 domain of Her2, and each of the light chains, or each of the heavy chains, contain an N-terminally linked polypeptide comprising an scFv polypeptide chain constituted of a heavy and light variable region linked by an scFv linker chain, and the scFv polypeptide chain is linked to the N terminus of the immunoglobulin heavy or light chain via an interdomain amino acid linker consisting of 1 to 20 amino acids.

In certain embodiments,
the first polypeptide chain has a sequence identity of 70% or more, particularly 80% or more, more particularly 90% or more, even more particularly 95% or more, with SEQ ID No. 1, wherein most particularly the first polypeptide chain is identical to SEQ ID No. 1, and
the second polypeptide chain has a sequence identity of 70% or more, particularly 80% or more, more particularly 90% or more, even more particularly 95% or more, with SEQ ID No. 2, wherein most particularly the second polypeptide chain is identical to SEQ ID No. 2.

In certain embodiments,
the first polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 1, and
the second polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 2.

In certain embodiments,
the third polypeptide chain has a sequence identity of 70% or more, particularly 80% or more, more particularly 90% or more, even more particularly 95% or more, with SEQ ID No. 1, wherein most particularly the third polypeptide chain is identical to SEQ ID No. 1, and
the fourth polypeptide chain has a sequence identity of 70% or more, particularly 80% or more, more particularly 90% or more, even more particularly 95% or more, with SEQ ID No. 2, wherein most particularly the fourth polypeptide chain is identical to SEQ ID No. 2.

In certain embodiments,
the third polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 1, and
the fourth polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 2.

In certain embodiments,
the first polypeptide chain and the third polypeptide chain each have a sequence identity of 70% or more, particularly 80% or more, more particularly 90% or more, even more particularly 95% or more, with SEQ ID No. 1, wherein most particularly the first polypeptide chain and the third polypeptide chain are identical to SEQ ID No. 1, and
the second polypeptide chain and the fourth polypeptide chain each have a sequence identity of 70% or more, particularly 80% or more, more particularly 90% or more, even more particularly 95% or more, with SEQ ID No. 2, wherein most particularly the second polypeptide chain and the fourth polypeptide chain are identical to SEQ ID No. 2.

In certain embodiments,
the first polypeptide chain and the third polypeptide chain each have a sequence identity of 70%, 71%, 72%, 73%, 74%, 75%, 76%, 77%, 78%, 79%, 80%, 81%, 82%, 97%, 98%, 99% or 100% with SEQ ID No. 1, and
the second polypeptide chain and the fourth polypeptide chain each have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 83%, 84%, 85%, 86%, 87%, 88%, 89%, 90%, 91%, 92%, 93%, 94%, 95%, 96%, 97%, 98%, 99% or 100% with SEQ ID No. 2.

The resulting tetrameric polypeptide is referred to as “441” (for identity). In this construct, IgG light chains (of the first and third polypeptide chain) comprising the VL antigen binding domain of antibody A21 are N-terminally fused to an scFv fragment comprising the VL and VH antigen binding domains of 4D5 (trastuzumab or HERCEPTIN, HER2 D4 binder) and combined with IgG heavy chains (the second and fourth polypeptide chains) comprising the VH antigen binding domain of antibody A21. The VL and VH antigen binding domains of A21 together constitute a HER2 D1 binder).

In certain embodiments,
the first polypeptide chain has a sequence identity of 70% or more, particularly 80% or more, more particularly 90% or more, even more particularly 95% or more, with SEQ ID No. 3, wherein most particularly the first polypeptide chain is identical to SEQ ID No. 3, and
the second polypeptide chain has a sequence identity of 70% or more, particularly 80% or more, more particularly 90% or more, even more particularly 95% or more, with SEQ ID No. 4, wherein most particularly the second polypeptide chain is identical to SEQ ID No. 4.

In certain embodiments,
the first polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 3, and
the second polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 4.

In certain embodiments,
the third polypeptide chain has a sequence identity of 70% or more, particularly 80% or more, more particularly 90% or more, even more particularly 95% or more, with SEQ ID No. 3, wherein most particularly the third polypeptide chain is identical to SEQ ID No. 3, and
the fourth polypeptide chain has a sequence identity of 70% or more, particularly 80% or more, more particularly 90% or more, even more particularly 95% or more, with SEQ ID No. 4, wherein most particularly the fourth polypeptide chain is identical to SEQ ID No. 4.

In certain embodiments,
the third polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 3, and
the fourth polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 4.

In certain embodiments,
the first polypeptide chain and the third polypeptide chain each have a sequence identity of 70% or more, particularly 80% or more, more particularly 90% or more, even more particularly 95% or more, with SEQ ID No. 3, wherein most particularly the first polypeptide chain and the third polypeptide chain are identical to SEQ ID No. 3, and
the second polypeptide chain and the fourth polypeptide chain each have a sequence identity of 70% or more, particularly 80% or more, more particularly 90% or more, even more particularly 95% or more, with SEQ ID No. 4, wherein most particularly the second polypeptide chain and the fourth polypeptide chain are identical to SEQ ID No. 4.

In certain embodiments,
the first polypeptide chain and the third polypeptide chain each have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 3, and
the second polypeptide chain and the fourth polypeptide chain each have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 4.

The resulting tetrameric polypeptide is referred to as “4702” (for identity). In this construct, IgG light chains (of the first and third polypeptide chain) comprising the VL antigen binding domain of antibody 702 are N-terminally fused to an scFv fragment comprising the VL and VH antigen binding domains of 4D5 (trastuzumab or HERCEPTIN, HER2 D4 binder) and combined with IgG heavy chains (the second and fourth polypeptide chains) comprising the VH antigen binding domain of antibody 702. The VL and VH antigen binding domains of 702 together constitute a HER2 D1 binder).

In certain embodiments,
the first polypeptide chain has a sequence identity of 70% or more, particularly 80% or more, more particularly 90% or more, even more particularly 95% or more, with SEQ ID No. 11, wherein most particularly the first polypeptide chain is identical to SEQ ID No. 11, and
the second polypeptide chain has a sequence identity of 70% or more, particularly 80% or more, more particularly 90% or more, even more particularly 95% or more, with SEQ ID No. 12, wherein most particularly the second polypeptide chain is identical to SEQ ID No. 12.

In certain embodiments,
the first polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 11, and
the second polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 12.

In certain embodiments,
the third polypeptide chain has a sequence identity of 70% or more, particularly 80% or more, more particularly 90% or more, even more particularly 95% or more, with SEQ ID No. 11, wherein most particularly the third polypeptide chain is identical to SEQ ID No. 11, and
the fourth polypeptide chain has a sequence identity of 70% or more, particularly 80% or more, more particularly 90% or more, even more particularly 95% or more, with SEQ ID No. 12, wherein most particularly the fourth polypeptide chain is identical to SEQ ID No. 12.

In certain embodiments,
the third polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 11, and
the fourth polypeptide chain has a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 12.

In certain embodiments,
the first polypeptide chain and the third polypeptide chain each have a sequence identity of 70% or more, particularly 80% or more, more particularly 90% or more, even more particularly 95% or more, with SEQ ID No. 11, wherein most particularly the first polypeptide chain and the third polypeptide chain are identical to SEQ ID No. 11, and
the second polypeptide chain and the fourth polypeptide chain each have a sequence identity of 70% or more, particularly 80% or more, more particularly 90% or more, even more particularly 95% or more, with SEQ ID No. 12, wherein most particularly the second polypeptide chain and the fourth polypeptide chain are identical to SEQ ID No. 12.

In certain embodiments,
the first polypeptide chain and the third polypeptide chain each have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 11, and
the second polypeptide chain and the fourth polypeptide chain each have a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with SEQ ID No. 12.

The resulting tetrameric polypeptide is referred to as “241” (for identity). In this construct, IgG heavy chains (of the second and fourth polypeptide chain) comprising the VH antigen binding domain of antibody A21 are N-terminally fused to an scFv fragment comprising the VL and VH antigen binding domains of 4D5 (trastuzumab or HERCEPTIN, HER2 D4 binder) and combined with IgG light chains (the first and third polypeptide chains) comprising the VL antigen binding domain of antibody A21. The VL and VH antigen binding domains of A21 together constitute a HER2 D1 binder).

In certain embodiments, the VH antigen binding domain is selected from the VH antigen binding domain of A21 (particularly SEQ ID No. 40, 42, 51, 52 or 77) and the VH antigen binding domain of 7C2 (particularly SEQ ID No. 79), and wherein the VL antigen binding domain is selected from the VL antigen binding domain of A21 (particularly SEQ ID No. 39, 41, 50 or 76) and the VL antigen binding domain of 7C2 (particularly SEQ ID No. 78).

In certain embodiments, the first and third polypeptide chains are identical to SEQ ID No. 1 or a functional equivalent peptide sequence having a sequence identity of at least 70%, 75%, 80%, 85%, 90%, 95%, 96%, 97%, 98% or 99%, and the second and fourth polypeptide chains are identical to SEQ ID No. 2 or a functional equivalent peptide sequence having a sequence identity of at least 70%, 75%, 80%, 85%, 90%, 95%, 96%, 97%, 98% or 99% (construct 441 in case of sequence identity).

In certain embodiments, the first and third polypeptide chains are identical to SEQ ID No. 3 or a functional equivalent peptide sequence having a sequence identity of at least 70%, 75%, 80%, 85%, 90%, 95%, 96%, 97%, 98% or 99%, and the second and fourth polypeptide chains are identical to SEQ ID No. 4 or a functional equivalent peptide sequence having a sequence identity of at least 70%, 75%, 80%, 85%, 90%, 95%, 96%, 97%, 98% or 99% (construct 47C2 in case of sequence identity).

In certain embodiments, the first and third polypeptide chains are identical to SEQ ID No. 11 or a functional equivalent peptide sequence having a sequence identity of at least 70%, 75%, 80%, 85%, 90%, 95%, 96%, 97%, 98% or 99%, and the second and fourth polypeptide chains are identical to SEQ ID No. 12 or a functional equivalent peptide sequence having a sequence identity of at least 70%, 75%, 80%, 85%, 90%, 95%, 96%, 97%, 98% or 99% (construct 241 in case of sequence identity).

The interdomain amino acid linker is not restricted in amino acid composition but amino acids shown to contribute to linker flexibility are chosen in particular embodiments contemplated herein. The inventors have shown linkers to work that consist of G, S and/or T residues, for example repeats of (GGₘS) and (GGₘT) with m selected from 1 to 3, and the entire linker length not exceeding 20, 25, or even 30. Interdomain linkers as short as one or two amino acids have been shown to work. The first and the third polypeptide may comprise the same interdomain amino acid linker or different interdomain amino acid linkers.

In certain embodiments, the interdomain amino acid linker consists of 1, 2, 3, 4, 5, 6, 7, 8, 9, 10, 11, 12, 13, 14, 15, 16, 17, 18, 19, 20, 21, 22, 23, 24 or 25 amino acids.

With regard to the length and sequence composition of the interdomain amino acid linker, the inventors' results indicate that any linker having an equivalent length of a maximum of 25 amino acids that is not expected, because of structure prediction, to interfere with the solubility of the resulting protein, is expected to function.

The polypeptide according to any one of the preceding claims, wherein the interdomain amino acid linker comprises or consists of amino acids G, A, J, S, T, P, C, V, M and E, particularly wherein the interdomain amino acid linker comprises or consists of amino acids G, S, A and T.

In particular embodiments, the interdomain amino acid linker is (GGΣ)ₙ with n being an integer and n≥4 (particularly n is 4, 5, 6, 7 or 8), and with Σ selected from S and T.

In particular embodiments, the interdomain amino acid linker is (GGS)ₙ with n being an integer and n≥4 (particularly n is 4, 5, 6, 7 or 8).

In particular embodiments, the interdomain amino acid linker is (GGT)ₙ with n being an integer and n≥4 (particularly n is 4, 5, 6, 7 or 8).

In particular embodiments, the interdomain amino acid linker is (GEG)ₙ with n being an integer and n≥4 (particularly n is 4, 5, 6, 7 or 8), and with E selected from S and T.

In particular embodiments, the interdomain amino acid linker is (ΓΓΣ)ₙ with n being an integer and n≥4 (particularly n is 4, 5, 6, 7 or 8), and with each F independently from any other F being selected being from A, G and V, and Σ being selected from S and T.

Important considerations at the time of choosing the linker sequence have been solubility and flexibility. The skilled person will readily be able to vary this sequence in composition and length based on the teaching herein and the knowledge available on linker design, as exemplified by Chen et al., 2013, Advanced Drug Delivery Reviews, 65, 1357-1369 and Evers et al., 2006, Biochemistry, 45, 13183-13192.

In certain embodiments, the interdomain amino acid linker is characterized by an amino acid sequence (GGGGS)ₙ, with n being 1, 2, 3, 4 or 5.

In certain embodiments, the interdomain amino acid linker comprises or is a sequence characterized by one of SEQ ID No. 17, SEQ ID No. 55 to SQ ID No. 69 and SEQ ID No. 82 to SEQ ID 91.

In certain embodiments, the interdomain amino acid linker comprises or consists of a peptide sequence selected from one of SEQ ID No. 17, SEQ ID No. 55 to SEQ ID No. 69 and SEQ ID No. 82 to SEQ ID No. 91 or a functional equivalent peptide sequence having a sequence identity of at least 70%.

A second aspect of the invention relates to the polypeptide according to the first aspect for use in a method for the prevention or treatment of a malignant neoplastic disease associated with expression of HER2 (a HER2-positive cancer).

Similarly, a dosage form for the prevention or treatment of a malignant neoplastic disease associated with expression of HER2 is provided, comprising a tetrameric polypeptide of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

A third aspect of the invention relates to an isolated nucleic acid encoding at least one of the first polypeptide chain, the second polypeptide chain, the third polypeptide chain and the fourth polypeptide chain of the tetrameric polypeptide according to the first aspect of the invention. In particular, the isolated nucleic acid may be comprised in a plasmid for expression in a bacterial or a eukaryotic host cell. The nucleic acid sequences encoding the first, the second, the third and the fourth polypeptide may be provided on the same plasmid or on separate plasmids, i. e for co-expression in the same host.

A fourth aspect of the invention relates to a host cell which is adapted to produce at least one of the first polypeptide chain, the second polypeptide chain, the third polypeptide chain and the fourth polypeptide chain of the tetrameric polypeptide according to the first aspect of the invention. In particular, the host cell is a bacterial cell or a eukaryotic cell. More particularly, the host cell is a Chinese Hamster Ovary (CHO) cell.

In certain embodiments, the host cell comprises the isolated nucleic acid according to the third aspect of the invention, such that the host cell is able to produce at least one of the first polypeptide chain, the second polypeptide chain, the third polypeptide chain and the fourth polypeptide chain of the polypeptide according to the first aspect of the invention. In particular, the first, second, third and fourth polypeptide may be co-produced in the same cell or produced separately and combined in vitro.

A fifth aspect of the invention relates to a method for obtaining the polypeptide according to the first aspect of the invention, wherein the method comprises culturing the host cell according to the fourth aspect of the invention, so that at least one of the first polypeptide chain, the second polypeptide chain, the third polypeptide chain and the fourth polypeptide chain of the polypeptide according to the first aspect of the invention is produced.

Pharmaceutical Composition and Administration

Another aspect of the invention relates to a pharmaceutical composition comprising a tetrameric polypeptide of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the tetrameric polypeptide of the present invention is typically formulated into pharmaceutical dosage forms to provide an easily controllable dosage of the drug and to give the patient an elegant and easily handleable product.

In embodiments of the invention relating to topical uses of the tetrameric polypeptide of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

The invention further relates to the following items, which may also be formulated as claims:

Item 1: A tetrameric polypeptide comprising or consisting of
a first polypeptide chain comprising a first ligand that binds to a HER2 D4 epitope, an interdomain amino acid linker and a first immunoglobulin domain,
a second polypeptide chain comprising a second immunoglobulin domain, wherein the first immunoglobulin domain and the second immunoglobulin domain together constitute a second ligand, particularly an Fab domain, that binds to a HER2 D1 epitope,
a third polypeptide chain comprising a third ligand that binds to a HER2 D4 epitope, an interdomain amino acid linker and a third immunoglobulin domain, and
a fourth polypeptide chain comprising a fourth immunoglobulin domain, wherein the third immunoglobulin domain and the fourth immunoglobulin domain together constitute a fourth ligand, particularly an Fab domain, that binds to a HER2 D1 epitope.

Item 2: The polypeptide according to item 1, wherein said first immunoglobulin domain is substantially the same as said third immunoglobulin domain, and/or wherein said second immunoglobulin domain is substantially the same as said fourth immunoglobulin domain.

Item 3: The polypeptide according to item 1 or 2, wherein said first ligand is substantially the same as said third ligand.

Item 4: The polypeptide according to any one of the preceding items, wherein said first ligand and/or said third ligand comprises or consists of a single-chain variable fragment polypeptide chain comprising an scFv heavy chain, an scFv linker chain, and an scFv light chain.

Item 5: The polypeptide according to item 4, wherein said scFv heavy chain is the VH domain of 4D5, and wherein said scFv light chain is the VL domain of 4D5.

Item 6: The polypeptide according to any one of the items 1 to 5, wherein
said first immunoglobulin domain is a VL domain, and said second immunoglobulin domain is a VH domain and/or wherein said third immunoglobulin domain is a VL domain, and said fourth immunoglobulin domain is a VH domain;or
said first immunoglobulin domain is a VH domain, and said second immunoglobulin domain is a VL domain and/or wherein said third immunoglobulin domain is a VH domain, and said fourth immunoglobulin domain is a VL domain.

Item 7: The polypeptide according to item 6, wherein said VH domain is C-terminally linked to a CH1 domain.

Item 8: The polypeptide according to item 7, wherein said CH1 domain is C-terminally linked to a CH2 domain or a CH2 domain and a CH3 domain.

Item 9: The polypeptide according to any one of the items 6 to 8, wherein said VL domain is C-terminally linked to a CL domain.

Item 10: The polypeptide according to any one of the items 6 to 9, wherein said VH domain is selected from the VH domain of A21 and the VH domain of 7C2, and wherein said VL domain is selected from the VL domain of A21 and the VL domain of 7C2.

Item 11: The polypeptide according to any one of the preceding items, wherein said interdomain amino acid linker consists of 1, 2, 3, 4, 5, 6, 7, 8, 9, 10, 11, 12, 13, 14, 15, 16, 17, 18, 19 or 20 amino acids.

Item 12: The polypeptide according to any one of the preceding items, wherein said interdomain amino acid linker comprises or consists of amino acids G, A, J, S, T, P, C, V, M and E, particularly wherein said interdomain amino acid linker comprises or consists of amino acids G, S, A and T.

Item 13: The polypeptide according to any one of the preceding items, wherein said interdomain amino acid linker is characterized by an amino acid sequence (GGGGS)ₙ, with n being 1, 2, 3, 4 or 5.

Item 14: The bispecific HER2-polypeptide according to any one of the preceding items, wherein said interdomain amino acid linker comprises or is a sequence characterized by one of SEQ ID 17, SEQ ID 55 to SQ ID 69 and SEQ ID 82 to SEQ ID 91.

Item 15: The polypeptide according to any one of the preceding items, wherein said interdomain amino acid linker comprises or consists of a peptide sequence selected from one of SEQ ID 17, SEQ ID 55 to SQ ID 69 and SEQ ID 82 to SEQ ID 91 or a functional equivalent peptide sequence having a sequence identity of at least 70%.

Item 16: The polypeptide according to any one of the preceding items for use in a method for the prevention or treatment of a malignant neoplastic disease associated with expression of Her2.

Wherever alternatives for single separable features such as, for example, an isotype protein or coding sequence, ligand type or medical indication are laid out herein as “embodiments”, it is to be understood that such alternatives may be combined freely to form discrete embodiments of the invention disclosed herein. Thus, any of the alternative embodiments for a detectable label may be combined with any of the alternative embodiments of ligand and these combinations may be combined with any medical indication or diagnostic method mentioned herein.

The invention is further illustrated by the following examples and figures, from which further embodiments and advantages can be drawn. These examples are meant to illustrate the invention but not to limit its scope.

### DESCRIPTION OF THE FIGURES

FIG. 1 shows schemes of biparatopic anti-HER2 binding agents.

FIG. 2 shows further schemes of biparatopic anti-HER2 binding agents

FIG. 3 shows a vector map of a plasmid for co-expression of the light and heavy chain of construct 441 in CHO cells (Pymex10 based vector with double expression cassette [CMV GOI polyA]).

FIG. 4 shows a vector map of a plasmid for co-expression of the light and heavy chain of construct 47C2 in CHO cells (Pymex10 based vector with double expression cassette [CMV GOI polyA]).

FIG. 5 shows a vector map of a plasmid for expression of construct 841 in CHO cells.

FIG. 6 shows an elution profile of construct 441 from Protein A affinity chromatography.

FIG. 7 shows an elution profile of construct 441 from cation exchange chromatography.

FIG. 8 shows an elution profile of construct 441 from size exclusion chromatography.

FIG. 9 shows a Coumassie-stained SDS-PAGE gel of fractions from purification of construct 441.

FIG. 10 shows the viability testing of CHOs during the expression of construct 441 (scFV-IgG). Expression optimization of construct 441 in CHOs cells for indicated time. Cells were cultured in CHOgro medium from Mlrus (MIR 6260) and additionally fed with free cysteine (reduced form) (2), glutathione (3), fetal calf serum (4) or all additives respectively (5). CHO cells were analyzed on CASY cell counter (Scharfe System).

FIG. 11 shows a Western blot of construct 441 expression, secreted to the medium of CHO cells after indicated times. Cells were cultured in CHOgro medium from Mlrus (MIR 6260) (1) and additionally fed with free cysteine (reduced form) (2), glutathione (3), fetal calf serum (4) or all additives together (5), respectively. Protein was precipitated from medium by acetone precipitation and re-solubilized in SDS PAGE buffer. Proteins were resolved on 4-12% gradient gel and the western blot was analyzed on an Odyssey system (LI-COR). Purified intact full length construct 441 is shown as control (A) and runs above the 170 kDa marker. Molecular weight marker Page ruler from Thermo Scientific is shown in red.

FIG. 12 shows cell proliferation assays (XTT) with BT474 cells after 4 days of treatment. Trastuzumab (TZB), biparatopic DARPin (6L1G) and different fusion variants of the biparatopic construct. LF IgG HL (murine parent of construct 441), HF IgG HL (murine parent of construct 241) show similar anti-proliferative activity compared to the biparatopic DARPin 6L1G, which is superior to trastuzumab (TZB). HF IgG LH (murine variant, no seq.) and LF IgG LH (murine variant, no seq.) show reduced anti-proliferative activity compared to biparatopic DARPin and higher IC₅₀ concentrations.

FIG. 13 shows cell proliferation assays (XTT) with BT474 cells after 4 days of treatment to test the effect of the linker length. Biparatopic DARPin (6L1G) and different fusion linker variants of the biparatopic construct (murine parent construct of 441) are compared. The 2-AA linker (GS) shows highest anti-proliferative activity. The 4-, 7- and 12-AA linkers show similar activity. The 22-AA linker variant shows reduced activity.

FIG. 14 shows cell proliferation assays (XTT) with BT474 cells after 4 days of treatment. Biparatopic DARPin (6G; 6L1G), biparatopic construct 441 (441), biparatopic construct 411 (humanized kappal VH1) and biparatopic construct 443 (humanized kappa4 VH3). All show similar plateau levels of anti-proliferative activity, except 443, which shows reduced activity.

FIG. 15 shows cell proliferation assays (XTT) with BT474 cells after 4 days of treatment with different humanized versions of A21 IgG, when fused to TZB scFv. The strategy of humanization is described above. Different variants use humanized kappal VH3 or a humanized kappal VH core graft.

FIG. 16 shows XTT cell proliferation assay with BT474 cells after 4 day of treatment. Tetravalent IgG (HF IgG HL and LF IgG HL murine) versus bivalent Fab fusions (HF Fab HL and LF Fab HL murine). All constructs show similar plateau and IC50 values.

FIG. 17 shows XTT cell proliferation assay with SKBR3 cells after 4 day of treatment. Biparatopic DARPin (6G) biparatopic construct (441 tf), trastuzumab (TZB).

FIG. 18 shows cell proliferation assays (XTT) with CALU-3 cells after 4 days of treatment. Biparatopic DARPin (6G), biparatopic construct (construct 441 (441tf), trastuzumab (TZB).

FIG. 19 shows cell proliferation assays (XTT) with BT474 cells after 4 days of treatment, testing effect of domain 1 binding unit. Biparatopic construct with A21 (construct 441tf) or 7C2 fusions show different IC50 and plateau level.

FIG. 20 shows cell proliferation assays (XTT) with BT474 cells after 4 days of treatment, testing the effect of domain 1 binding unit. Biparatopic construct with A21 (construct 441) or with 39S (39s HF IgG H)L

FIG. 21 shows XTT cell proliferation assays with HCC1419 cells after 4 days of treatment. Biparatopic DARPin (6G; 6L1G), biparatopic construct 441 (441tf) and bivalent LF-oaFabFc (A21-TZB-40a). 441 and 6G show similar inhibition of cell proliferation after 4 days. LF-oaFabFc show slightly reduced inhibition of cell proliferation compared to 441.

FIG. 22 shows XTT cell proliferation assay with BT474 and HCC1419 cells after 4 day of treatment. All human.

FIG. 23 shows XTT cell proliferation assay with BT474 and HCC1419 cells after 4 day of treatment. All human.

FIG. 24 shows a) in the upper panel XTT cell proliferation assays with BT474 (left) and HCC1419 (right) cells after 4 day of treatment; and in the lower panel XTT cell proliferation assays with BT474 (left) and HCC1419 (right) cells after 4 day of treatment (variants with higher affinity (NGS and GGG)); b) repeated experiments with a new expression of NGS.

FIG. 25 shows XTT cell proliferation assays with BT474 (left) and HCC1419 (right) cells after 4 day of treatment.

FIG. 26 shows XTT cell proliferation assays with HCC1419 cells grown as 3D spheroids.

FIG. 27 shows Western Blots 24 hours post treatment (BT474) with indicated agents (murine).

FIG. 28 shows in the upper panel Induction of apoptosis in BT474 cells after 3 days of treatment. Average number of propidium iodide (PI) positive cells was determined for 4 replicates, counted by cell profiler and was analyzed with Student's t-test. Biparatopic construct (441, 441tf) induced significantly more cell death than trastuzumab (TZB). 441 and biparatopic DARPin (6L1G) show similar level of cell death; and in the lower panel Induction of apoptosis in BT474 cells after 3 days of treatment. Average number of annexin-V positive cells was determined for 3-4 replicates, counted by cell profiler and was analyzed with Student's t-test. Biparatopic construct 441 induced significantly more apoptosis than trastuzumab (TZB). Construct 441 and 6L1G show similar level of apoptosis.

FIG. 29 shows images of BT474 cells treated with the indicated agents for 3 days.

FIG. 30 shows Alexa647-labeled trastuzumab (TZB), biparatopic construct 441 and biparatopic one armed constructs oaLF and oaHF were incubated for 1 h at 100 nM concentration with 3 million BT474 cells in 3 ml PBS containing NaN₃ (0.1%) and BSA (1%) at 4° C. Note that BT474 cells were pre-treated with 0.1% NaN₃ in PBS with 1% BSA to block internalization before binding. Cells were analyzed afterwards on CyFlow Space instrument (Partec). All binding agents show specific binding to the surface of HER2-positive BT474 cells.

FIG. 31 shows the induction of cell death after treatment with 100 nM of indicated agents. BT474, N87, HCC1419 and SKBR3 cells were seeded 24 h before treatment in 96 black clear-well microscopy plates (Nunc), continuously treated for 3 days and stained with HOECHST-33342 (Invitrogen) for total cells and with propidium iodide (Sigma) for membrane-permeable dead cells. Cells were analyzed on a Lionheart FX Automated Microscope (BioTek Instruments) and the number of propidium iodide and HOECHST-33342 positive cells was quantified with Gen5 software (BioTek Instruments). The ratio of propidium iodide and HOECHST-33342 positive cells was calculated for 3 biological replicates and the mean and SD is shown in the corresponding column plots. Biparatopic binding agents (6L1G, 441, 841, LFoa, 241, 641, HFoa, 7C2LF) binding to domain 1 and 4 of HER2 induce continuously more dead cells than trastuzumab (TZB) or the combination of trastuzumab and pertuzumab (TZB+PZB) in HER2-positive cancer cells.

FIG. 32 shows the half-life of construct 441 in the serum of NSG mice. Drawn sera of mice with previous 441 injections (3 mg/kg) were analyzed by sandwich ELISA. 441 showed an alpha phase of around 4.3 hours, followed by a beta phase of more than 45 hours.

FIG. 33 shows in-vivo activity of 441 on N87 xenografts in SCID beige mice. After N87 tumors had reached 150 mm³ in size, mice were treated with eight injections of 441 (10 mg/kg) during four weeks. 441 lead to significant tumor size reduction compared to untreated mice and TZB (10 mg/kg) or huA21G (10 mg/kg) treated mice.

FIG. 34 shows representative microscopy images of BT-474 cells after treatment for 2 h with trastuzumab (TZB), huA21G (A21), their combination, or 441, and non-treated cells, either without or with addition of an anti-human primary antibody, as controls. Nuclei were stained using 2-(4-amidinophenyl)-1H-indo1-6-carboximidamide (DAPI), antibodies were detected with an anti-human Fc antibody from goat, and lysosomal compartments using an anti-LAMP1 antibody.

FIG. 35 shows the result of a time-course treatment and subsequent surface protein internalization and degradation assay for the constructs 441 and 841, hA21G, trastuzumab (TZB), the combination of trastuzumab and hA21G (TZB+hA21G), pertuzumab (PZB), the combination of trastuzumab and pertuzumab (TZB+PZB), and the inhibitor of HSP90, geldanamycin (GA).

Sequence listings:
SEQ ID No. 1 (“441 polypeptide 1”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSDIVLTQSPDSLAVSLGERATINCRSSQTLLYSNNQKNYLAWYQKKPGQPPKLLI
SWAFTRKSGVPDRFSGSGSGTDFTLTISSLQAEDVAVYYCQQYSNYPWTFGQGTKVEIKRT
VAAPSVFIFPPSDEQLKSGTASVKCLLNNFYPREAKVQWKVDNALQSGNSQESVTEQDSKD
STYSLSSTLTLSKADYEKHKVYACEVTHQGLSSPVTKSFNRGEC
SEQ ID No. 2 (“441 polypeptide 2”):
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 3 (“47C2 polypeptide 1”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSDIVMTQSPDSLAVSLGERATINCRASQSVSGSRFTYMHWYQQKPGQPPKLLI
KYASILESGVPDRFSGSGSGTDFTLTISSLQAEDVAVYYCQHSWEIPPWTFGQGTKVEIKRT
VAAPSVFIFPPSDEQLKSGTASVKCLLNNFYPREAKVQWKVDNALQSGNSQESVTEQDSKD
STYSLSSTLTLSKADYEKHKVYACEVTHQGLSSPVTKSFNRGEC
SEQ ID No. 4 (“47C2 polypeptide 2”):
EVQLVQSGAEVKKPGASVKVSCKASGYSFTGYWMNWVRQAPGQGLEWIGMIHPLDAEIRA
NQKFRDRVTITVDTSTSTAYLELSSLRSEDTAVYYCARGTYDGGFEYWGQGTLVTVSSAST
KGPSVFPLAPSSKSTSGGTAALGCLVKDYFPEPVTVSWNSGALTSGVHTFPAVLQSSGLYS
LESVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFS
CSVMHEALHNHYTQKSLSLSK
SEQ ID No. 5 (“841 polypeptide 2”):
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSC
SEQ ID No. 6 (“87C2 polypeptide 2”):
EVQLVQSGAEVKKPGASVKVSCKASGYSFTGYWMNWVRQAPGQGLEWIGMIHPLDAEIRA
NQKFRDRVTITVDTSTSTAYLELSSLRSEDTAVYYCARGTYDGGFEYWGQGTLVTVSSAST
KGPSVFPLAPSSKSTSGGTAALGCLVKDYFPEPVTVSWNSGALTSGVHTFPAVLQSSGLYS
LESVVTVPSSSLGTQTYICNVNHKPSNTKVDKRVEPKSC
SEQ ID No. 7 (“841Fc polypeptide 1”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSDIVLTQSPDSLAVSLGERATINCRSSQTLLYSNNQKNYLAWYQKKPGQPPKLLI
SWAFTRKSGVPDRFSGSGSGTDFTLTISSLQAEDVAVYYCQQYSNYPWTFGQGTKVEIKRT
VAAPSVFIFPPSDEQLKSGTASVKCLLNNFYPREAKVQWKVDNALQSGNSQESVTEQDSKD
STYSLSSTLTLSKADYEKHKVYACEVTHQGLSSPVTKSFNRGECDKTHTCPPCPAPELLGG
PSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRKEMTK
NQVSLTCLVKGFYPSDIAVEWESNGQPENNYKTTPPVLKSDGSFFLYSKLTVDKSRWQQG
NVFSCSVMHEALHNHYTQKSLSLSPGK
SEQ ID No. 8 (“841Fc polypeptide 2”):
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYDTTPPVLDSDGSFFLYSDLTVDKSRWQQGNV
FSCSVMHEALHNHYTQKSLSLSPGK
SEQ ID No. 9 (“87C2Fc polypeptide 1”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSDIVMTQSPDSLAVSLGERATINCRASQSVSGSRFTYMHWYQQKPGQPPKLLI
KYASILESGVPDRFSGSGSGTDFTLTISSLQAEDVAVYYCQHSWEIPPWTFGQGTKVEIKRT
VAAPSVFIFPPSDEQLKSGTASVKCLLNNFYPREAKVQWKVDNALQSGNSQESVTEQDSKD
STYSLSSTLTLSKADYEKHKVYACEVTHQGLSSPVTKSFNRGECDKTHTCPPCPAPELLGG
PSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRKEMTK
NQVSLTCLVKGFYPSDIAVEWESNGQPENNYKTTPPVLKSDGSFFLYSKLTVDKSRWQQG
NVFSCSVMHEALHNHYTQKSLSLSPGK
SEQ ID No. 10 (“87C2Fc polypeptide 2”):
EVQLVQSGAEVKKPGASVKVSCKASGYSFTGYWMNWVRQAPGQGLEWIGMIHPLDAEIRA
NQKFRDRVTITVDTSTSTAYLELSSLRSEDTAVYYCARGTYDGGFEYWGQGTLVTVSSAST
KGPSVFPLAPSSKSTSGGTAALGCLVKDYFPEPVTVSWNSGALTSGVHTFPAVLQSSGLYS
LESVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVS
LTCLVKGFYPSDIAVEWESNGQPENNYDTTPPVLDSDGSFFLYSDLTVDKSRWQQGNVFS
CSVMHEALHNHYTQKSLSLSPGK
SEQ ID No. 11 (“241 polypeptide 1”):
DIVLTQSPDSLAVSLGERATINCRSSQTLLYSNNQKNYLAWYQKKPGQPPKLLISWAFTRKS
GVPDRFSGSGSGTDFTLTISSLQAEDVAVYYCQQYSNYPWTFGQGTKVEIKRTVAAPSVFIF
PPSDEQLKSGTASVKCLLNNFYPREAKVQWKVDNALQSGNSQESVTEQDSKDSTYSLSST
LTLSKADYEKHKVYACEVTHQGLSSPVTKSFNRGEC
SEQ ID No. 12 (“241 polypeptide 2”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSQVQLVQSGAEVKKPGASVKVSCKASGYSFTGYFINWVREAPGQGLEWMGHI
SSSYATSTYNQKFQGRVTFTVDTSSSTAYMELSSLRSEDTAVYYCVRSGNYEEYAMDYWG
QGTLVTVSSASTKGPSVFPLAPSSKSTSGGTAALGCLVKDYFPEPVTVSWNSGALTSGVHT
FPAVLQSSGLYSLESVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVD
KSRWQQGNVFSCSVMHEALHNHYTQKSLSLSK
SEQ ID No. 13 (“641 polypeptide 2”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSQVQLVQSGAEVKKPGASVKVSCKASGYSFTGYFINWVREAPGQGLEWMGHI
SSSYATSTYNQKFQGRVTFTVDTSSSTAYMELSSLRSEDTAVYYCVRSGNYEEYAMDYWG
QGTLVTVSSASTKGPSVFPLAPSSKSTSGGTAALGCLVKDYFPEPVTVSWNSGALTSGVHT
FPAVLQSSGLYSLESVVTVPSSSLGTQTYICNVNHKPSNTKVDKRVEPKSC
SEQ ID No. 14 (“scFv TZB L chain”):
DIQMTQSPSSLSASVGDRVTITCRASQDVNTAVAWYQQKPGKAPKLLIYSASFLYSGVPSRF
SGSRSGTDFTLTISSLQPEDFATYYCQQHYTTPPTFGQGTKVEIK
SEQ ID No. 15 (“scFv TZB H chain”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
SEQ ID No. 16 (“scFv TZB linker”):
GGGGSGGGGSGGGGSGGGGS
SEQ ID No. 17 (“B1 to B2 linker (link1)”):
GS
SEQ ID No. 18 (“A21 L chain”)
DIVLTQSPDSLAVSLGERATINCRSSQTLLYSNNQKNYLAWYQKKPGQPPKLLISWAFTRKS
GVPDRFSGSGSGTDFTLTISSLQAEDVAVYYCQQYSNYPWTFGQGTKVEIKRTVAAPSVFIF
PPSDEQLKSGTASVKCLLNNFYPREAKVQWKVDNALQSGNSQESVTEQDSKDSTYSLSST
LTLSKADYEKHKVYACEVTHQGLSSPVTKSFNRGEC
SEQ ID No. 19 (“A21 H chain with Fc”)
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 20 (“A21 H chain for Fab”)
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSC
SEQ ID No. 21: (“TZB CDR H1”)
GFNIKDTYIH
SEQ ID No. 22: (“TZB CDR H2”)
RIYPTNGYTRYADSVKG
SEQ ID No. 23: (“TZB CDR H3”)
WGGDGFYAMDY
SEQ ID No. 24: (“TZB CDR L1”)
RASQDVNTAVA
SEQ ID No. 25: (“TZB CDR L2”)
SASFLYS
SEQ ID No. 26: (“TZB CDR L3”)
QQHYTTPPT
SEQ ID No. 27: (“A21 CDR H1”)
GYSFTGYFIN
SEQ ID No. 28: (“A21 CDR H2”)
HISSSYATSTYNQKFQG
SEQ ID No. 29 (“A21 CDR H3”)
SGNYEEYAMDY
SEQ ID No. 30: (“A21 CDR L1”)
RSSQTLLYSNNQKNYLA
SEQ ID No. 31: (“A21 CDR L2”)
WAFTRKS
SEQ ID No. 32: (“A21 CDR L3”)
QQYSNYPWT
SEQ ID No. 33: (“7C2 CDR H1”)
GYSFTGYWMN
SEQ ID No. 34: (“7C2 CDR H2”)
MIHPLDAEIRANQKFR
SEQ ID No. 35: (“7C2 CDR H3”)
GTYDGGFEY
SEQ ID No. 36: (“7C2 CDR L1”)
RASQSVSGSRFTYMH
SEQ ID No. 37: (“7C2 CDR L2”)
YASILES
SEQ ID No. 38: (“7C2 CDR L3”)
QHSWEIPPWT
SEQ ID No. 39 (“A21 L chain V1”)
DIVLTQSPDSLAVSLGERATINCRSSQTLLYSNNQKNYLAWYQQKPGQPPKLLISWAFTRKS
GVPDRFSGSGSGTDFTLTISSLQAEDVAVYYCQQYSNYPWTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID No. 40: (“A21 H chain V1”)
QVQLVQSGAEVKKPGASVKVSCKASGYSFTGYFINWVRQAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSC
SEQ ID No. 41: (“A21 L chain V2”)
DIVLTQSPDSLAVSLGERATINCRSSQPLEYSNNQWNYLAWYQKKPGQPPKLLISWAFTRK
SGVPDRFSGSGSGTDFTLTISSLQAEDVAVYYCGQYSDYPNTFGQGTKVEIKRTVAAPSVFI
FPPSDEQLKSGTASVKCLLNNFYPREAKVQWKVDNALQSGNSQESVTEQDSKDSTYSLSS
TLTLSKADYEKHKVYACEVTHQGLSSPVTKSFNRGEC
SEQ ID No. 42: (“A21 H chain V2”)
QVQLVQSGAEVKKPGASVKVSCKASGYPFTQYFIHWVREAPGQGLEWMGHISSSYATVDY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 43: (“scFv TZB L chain V1”)
DIQMTQSPSSLSASVGDRVTITCRASQDVSTAVAWYQQKPGKAPKLLIYSASFLYSGVPSRF
SGSRSGTDFTLTISSLQPEDFATYYCQQHYTTPPTFGQGTKVEIK
SEQ ID No. 44: (“scFv TZB H chain V1”)
EVQLVESGGGLVQPGGSLRLSCAASGFNIKDTYIHWVRQAPGKGLEWVARIYPTNAYTRYA
DSVKGRFTISADTSKNTAYLQMNSLRAEDTAVYYCSRWGGTGFYAMDYWGQGTLVTVSS
SEQ ID No. 45: (“Alternative Fc part - no mut”)
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID No. 46: (“Alternative Fc part - Knob into hole - knob site V1”)
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSREEMTKNQVSLYCLVKGFYPSDIAVEWESNGQPENNYKTTPPVLDSDGSFFLYSKLTVD
KSRWQQGNVFSCSVMHEALHNHYTQKSLSLSPGK
SEQ ID No. 47: (“Alternative Fc part - Knob into hole - hole site V1”)
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLTSKLTVD
KSRWQQGNVFSCSVMHEALHNHYTQKSLSLSPGK
SEQ ID No. 48: (“Alternative Fc part - Knob into hole - knob site V2”)
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PCREEMTKNQVSLWCLVKGFYPSDIAVEWESNGQPENNYKTTPPVLDSDGSFFLYSKLTV
DKSRWQQGNVFSCSVMHEALHNHYTQKSLSLSPGK
SEQ ID No. 49: (“Alternative Fc part - Knob into hole - hole site V2”)
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVCTLP
PSREEMTKNQVSLSCAVKGFYPSDIAVEWESNGQPENNYKTTPPVLDSDGSFFLVSKLTVD
KSRWQQGNVFSCSVMHEALHNHYTQKSLSLSPGK
SEQ ID No. 50: (“A21 L chain V3”)
DIQLTQSPSSLSASVGDRVTITCRSSQTLLYSNNQKNYLAWYQQKPGKAPKLLISWAFTRKS
GVPSRFSGSGSGTDFTLTISSLQPEDFATYYCQQYSNYPWTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID No. 51: (“A21 H chain V3”)
EVQLVQSGPELVQPGGSVRISCAASGYSFTGYFINWVKQAPGKGLEWISHISSSYATSTYN
QSFKGRATFSVDTSSSTAYMQLNSLRAEDTAVYYCVRSGNYEEYAMDYWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSC
SEQ ID No. 52: (“A21 H chain V4”)
VQLVESGGGLVQPGGSLRLSCAASGYSFTGYFINWVRQAPGKGLEWVSHISSSYATSTYN
QSVKGRFTFSVDTSSSTAYLQMNSLRAEDTAVYYCVRSGNYEEYAMDYWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSC
SEQ ID No. 53: (“TZB H chain V2”)
QVQLVQSGAEVKKPGASVKVSCKASGFNIKDTYIHWVRQAPGQGLEQMGRIYPTNGYTRY
DPKFQGRVTITADTSSNTAYMELSSLRSEDTAVYYCSRWGGDGFYAMDYWGQGTLVTVSS
SEQ ID No. 54: (“TZB H chain V3”)
EVQLVQSGPELVQPGGSLRLSCAASGFNIKDTYIHWVKQAPGKGLEWISRIYPTNGYTRYD
PSFKGRATISADTSSNTAYLQVNSLRAEDTAVYYCSRWGGDGFYAMDYWGQGTLVTVSS
SEQ ID No. 55: (“Poly-Gly-linker”)
GGGGG
SEQ ID No. 56: (“Ova-linker”)
GSGSGS
SEQ ID No. 57: (“Trans-linker”)
GSGGGTGGGSG
SEQ ID No. 58: (“Pro-linker”)
PPP
SEQ ID No. 59: (“PAS-linker 1”)
ASPAAPAPASPAAPAPSAPAA
SEQ ID No. 60: (“PAS-linker 2”)
ASAAAPAAASAAASAPSAAAA
SEQ ID No. 61: (“PAS-linker 3”)
AASPAAPSAPPAAASPAAPSAPPAA
SEQ ID No. 62: (“PAS-linker 4”)
ASPASA
SEQ ID No. 63: (“PAS-linker 5”)
ASPASPASA
SEQ ID No. 64: (“XS-linker 1”)
PAGSP
SEQ ID No. 65: (“XS-linker 2”)
STEPS
SEQ ID No. 66: (“XS-linker 3”)
STEEG
SEQ ID No. 67: (“XS-linker 4”)
GSAPG
SEQ ID No. 68: (“Proper-linker1”)
GASTP
SEQ ID No. 69: (“Proper-linker2”)
GPSAT
SEQ ID No. 70: (“39s light chain”)
DIVMTQTPLSLSVTPGQPASISCKSSQSVFFRSNNKNILAWYLQKPGQPPQLLIYWASSRES
GVPDRFSGSGSGTDFTLKISRVEAEDVGVYYCQQYFGSPFTFGPGTKVDIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID No. 71: (“39s heavy chain”)
EVQLVESGGGLVKPGGSLRLSCAASGFTFSSYSMSWVRQAPGKGLEWVSSISSSSSYIYYA
DSVKGRFTISRDNAKNSLYLQMNSLRAEDTAVYYCARGGDAYNYYYFDYWGQGTLVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 72: (“H218 light chain”)
QSALTQPASVSGSPGQSITISCTGTSSDVGGYNYVSWYQQHPGKAPKLMIYDVSKRPSGVS
NRFSGSKSGNTASLTISGLQAEDEADYYCSSYTSSSTLVFGGGTKLTVLGTVAAPSVFIFPP
SDEQLKSGTASVVCLLNNFYPREAKVQWKVDNALQSGNSQESVTEQDSKDSTYSLSSTLTL
SKADYEKHKVYACEVTHQGLSSPVTKSFNRGEC
SEQ ID No. 73: (“H218 heavy chain”)
EVQLVQSGAEVKKPGESLKISCKGSGYSFTSYWIGWVRQAPGQGLEWMGWISAYNGNTN
YAQKLQGRVTMTTDTSTSTAYMELRSLRSDDTAVYYCAREGDGAFDYWGQGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFS
CSVMHEALHNHYTQKSLSLSK
SEQ ID No. 74: (“MF3958 light chain”)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPTFGQGTKVEIKRTVAAPSVFIFPPSDEQL
KSGTASVKCLLNNFYPREAKVQWKVDNALQSGNSQESVTEQDSKDSTYSLSSTLTLSKADY
EKHKVYACEVTHQGLSSPVTKSFNRGEC
SEQ ID No. 75: (“MF3958 heavy chain”)
QVQLVQSGAEVKKPGASVKLSCKASGYTFTAYYINWVRQAPGQGLEWIGRIYPGSGYTSYA
QKFQGRATLTADESTSTAYMELSSLRSEDTAVYFCARPPVYYDSAWFAYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 76: (“A21 L chain V4”)
DIVLTQSPDSLAVSLGERATINCRSSQTLLYSNNQKNYLAWYQKKPGQPPKLLISWAFTRKS
GVPDRFSGSGSGTDFTLTISSLQAEDVAVYYCQQYSNYPWTFGQGTKVEIKRTVAAPSVFIF
PPSDEQLKSGTASVKCLLNNFYPREAKVQWKVDNALQSGNSQESVTEQDSKDSTYSLSST
LTLSKADYEKHKVYACEVTHQGLSSPVTKSFNRGEC
SEQ ID No. 77: (“A21 H chain with Fc V2”)
QVQLVQSGAEVKKPGASVKVSCKASGYSFTGYFINWVREAPGQGLEWMGHISSSYATSTY
NQKFQGRVTFTVDTSSSTAYMELSSLRSEDTAVYYCVRSGNYEEYAMDYWGQGTLVTVSS
ASTKGPSVFPLAPSSKSTSGGTAALGCLVKDYFPEPVTVSWNSGALTSGVHTFPAVLQSSG
LYSLESVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 78: (“7C2 light chain”)
DIVMTQSPDSLAVSLGERATINCRASQSVSGSRFTYMHWYQQKPGQPPKLLIKYASILESGV
PDRFSGSGSGTDFTLTISSLQAEDVAVYYCQHSWEIPPWTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID No. 79: (“7C2 heavy chain”)
EVQLVQSGAEVKKPGASVKVSCKASGYSFTGYWMNWVRQAPGQGLEWIGMIHPLDAEIRA
NQKFRDRVTITVDTSTSTAYLELSSLRSEDTAVYYCARGTYDGGFEYWGQGTLVTVSSAST
KGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFS
CSVMHEALHNHYTQKSLSLSK
SEQ ID No. 80: (“TZB-HC”)
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVF
SCSVMHEALHNHYTQKSLSLSK
SEQ ID No. 81: (“TZB-LC”)
DIQMTQSPSSLSASVGDRVTITCRASQDVNTAVAWYQQKPGKAPKLLIYSASFLYSGVPSRF
SGSRSGTDFTLTISSLQPEDFATYYCQQHYTTPPTFGQGTKVEIKRTVAAPSVFIFPPSDEQL
KSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID 82 (“GS-linker 1”): GGGGS
SEQ ID 83 (“GS-linker 2”): GGGGSGGGGS
SEQ ID 84 (“GS-linker 3”): GGGGSGGGGSGGGGS
SEQ ID 85 (“GS-linker 4”): GGGGSGGGGSGGGGSGGGGS
SEQ ID 86 (“GS-linker 5”): GGGGSGGGGSGGGGSGGGGSGGGGS
SEQ ID 87 (“GA-linker”): GAAGAAG
SEQ ID 88 (“GT-linker”): GGTGGT
SEQ ID 89 (“ST-linker”): STSTS
SEQ ID 90 (“PA-linker”): PAPAP
SEQ ID 91 (“SA-linker”): SAASAAS
SEQ ID 92 (“HF-oaFabFc light chain”/“A21-TZB-2oa light chain”):
DIVLTQSPDSLAVSLGERATINCRSSQTLLYSNNQKNYLAWYQKKPGQPPKLLISWAFTRKS
GVPDRFSGSGSGTDFTLTISSLQAEDVAVYYCQQYSNYPWTFGQGTKVEIKRTVAAPSVFIF
PPSDEQLKSGTASVKCLLNNFYPREAKVQWKVDNALQSGNSQESVTEQDSKDSTYSLSST
LTLSKADYEKHKVYACEVTHQGLSSPVTKSFNRGESDKTHTCPPCPAPELLGGPSVFLFPP
KPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTC
LVKGFYPSDIAVEWESNGQPENNYDTTPPVLDSDGSFFLYSDLTVDKSRWQQGNVFSCSV
MHEALHNHYTQKSLSLSPGK
SEQ ID 93 (“HF-oaFabFc heavy chain”/“A21-TZB-2oa heavy chain”):
EVQLVESGGGLVQPGGSLRLSCAASGFNIKDTYIHWVRQAPGKGLEWVARIYPTNGYTRYA
DSVKGRFTISADTSKNTAYLQMNSLRAEDTAVYYCSRWGGDGFYAMDYWGQGTLVTVSS
GGGGSGGGGSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYCQQHYTTPPTFGQ
GTKVEIKGSQVQLVQSGAEVKKPGASVKVSCKASGYSFTGYFINWVREAPGQGLEWMGHI
SSSYATSTYNQKFQGRVTFTVDTSSSTAYMELSSLRSEDTAVYYCVRSGNYEEYAMDYWG
QGTLVTVSSASTKGPSVFPLAPSSKSTSGGTAALGCLVKDYFPEPVTVSWNSGALTSGVHT
FPAVLQSSGLYSLESVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSRKEMTKNQVSLTCLVKGFYPSDIAVEWESNGQPENNYKTTPPVLKSDGSFFLYSKLTVD
KSRWQQGNVFSCSVMHEALHNHYTQKSLSLSPGK

### EXAMPLE 1

### Biparatopic Anti-HER2 Binding Agents

The inventors have generated biparatopic IgG derivatives. In contrast to other available biparatopic HER2-targeting antibodies, e.g. the antibody-drug conjugate (ADC) from Medimmune MED14276 (Li et al., 2016), these IgGs show very strong anti-tumor activity as “naked” binding proteins, i.e., without attached drug (Kast et al., in preparation). Thus, it is believed that these novel biparatopic anti-HER2 IgGs combine the mechanisms of action of trastuzumab plus pertuzumab plus the action of small molecule kinases inhibitors against HER2 in one single molecule. In addition, potential off-target effects of the biparatopic anti-HER2 IgGs are expected to remain far below those of ADC fusions, such as T-DM1 or MED14276, as they can only act on HER2-addicted cells, while ADCs can via their toxin act in many healthy tissue. This opens up the therapeutic windows for new combination therapies. Furthermore, pan-ErbB inhibition by polymerization of HER2 receptors may passively block compensatory activation of other receptor tyrosine kinases (RTKs). The biparatopic anti-HER2 binding agents interfere with the free lateral movement of HER2 receptors on the cell surface of HER2-amplified cancer, yet without inducing signaling competent complexes, which may block the activation of other RTKs. Consequently, biparatopic anti-HER2 binding agents may show strong synergies with small molecule inhibitors, which tend to induce expression of compensatory RTKs that eventually drives escape from therapy. Therefore, biparatopic anti-HER2 IgGs bear a very high potential to elicit strong anti-tumor synergies in combination with small-molecule inhibitors on a broad panel of HER2-amplified cancers. The potential for synergies with small-molecule inhibitors is superior to current single-specificity antibodies or antibody combinations.

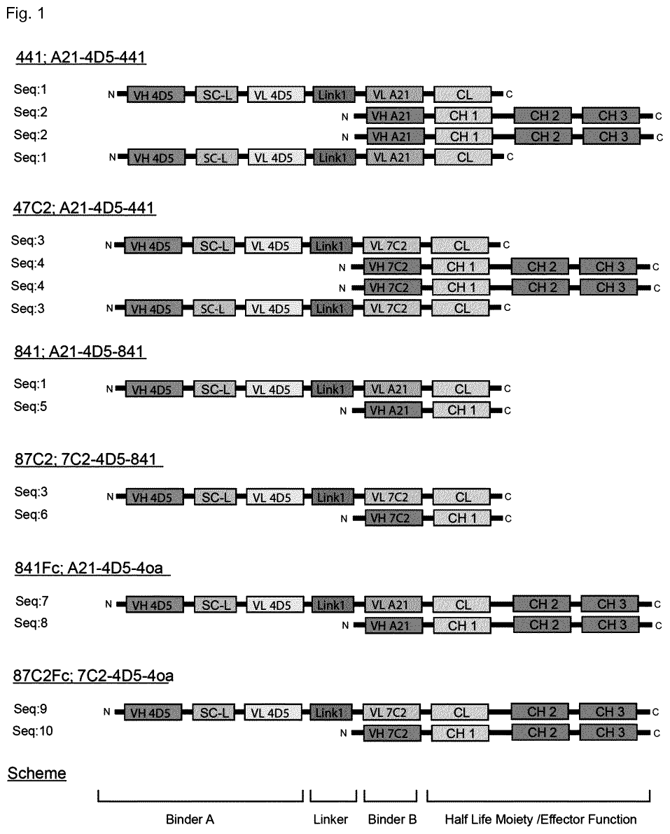

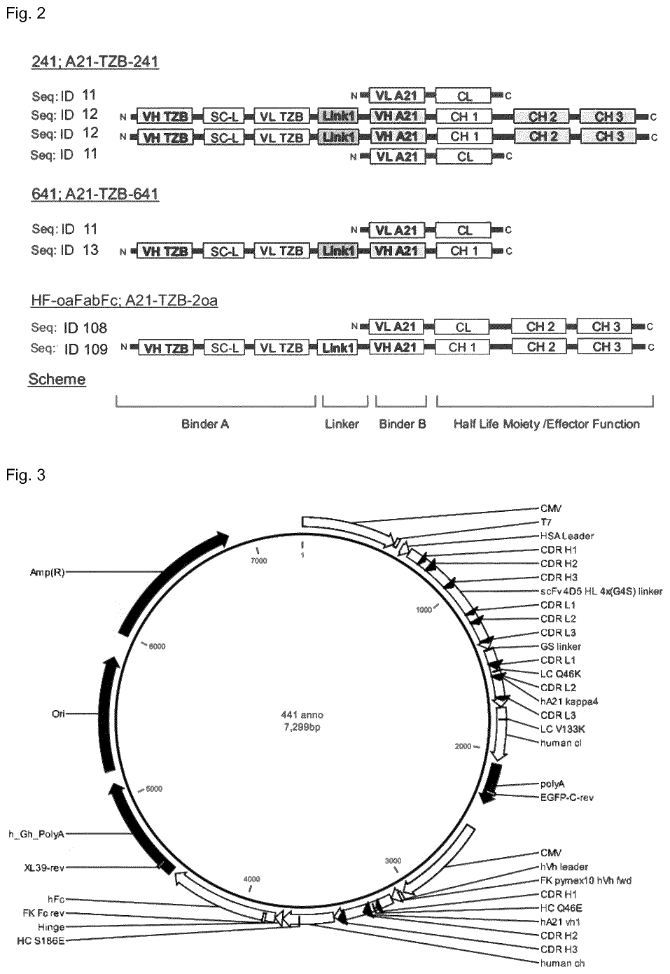

Illustrative schemes of preferred biparatopic IgG constructs are shown in FIGS. 1 and 2

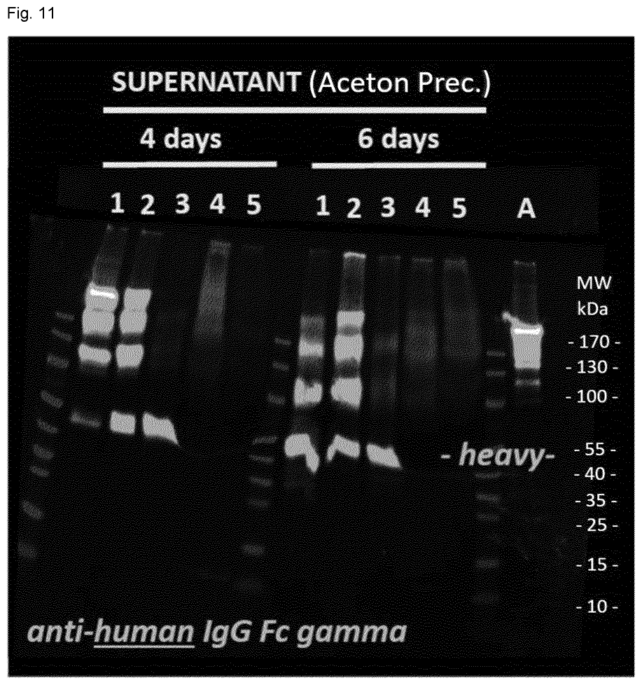

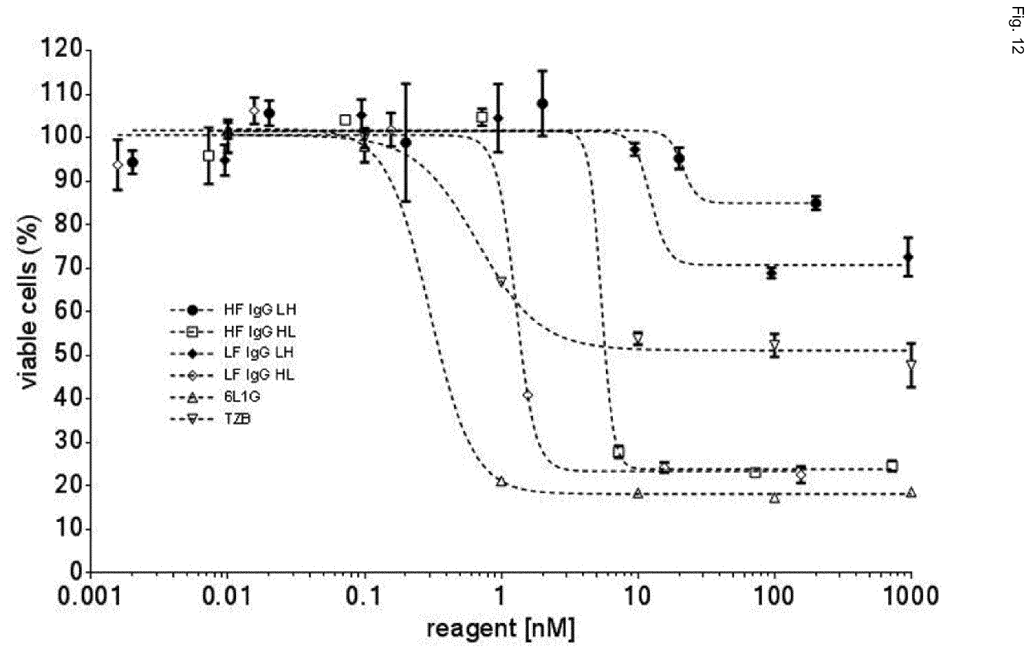

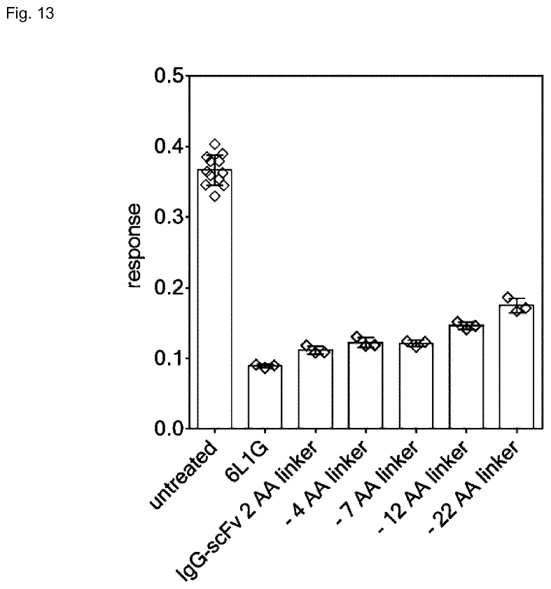

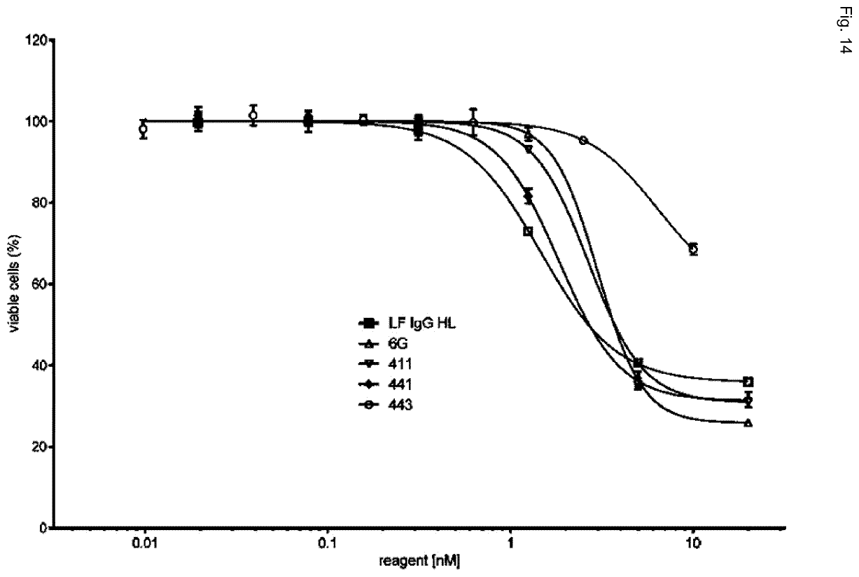

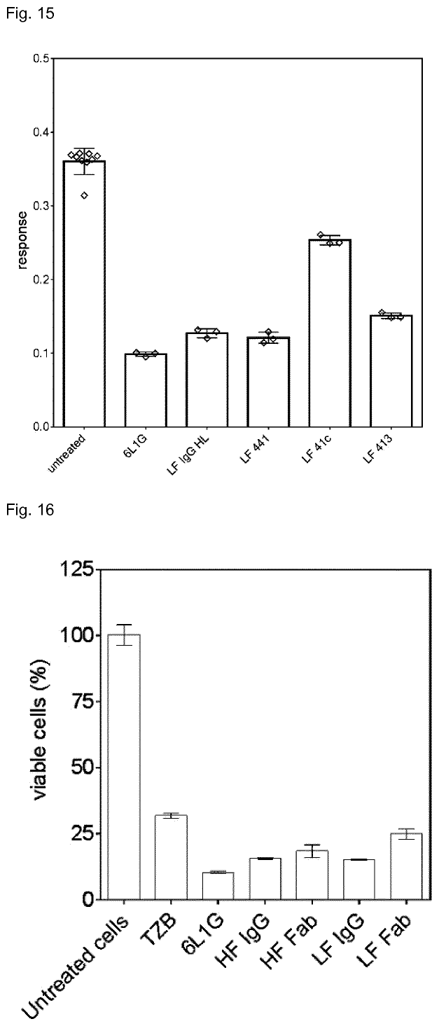

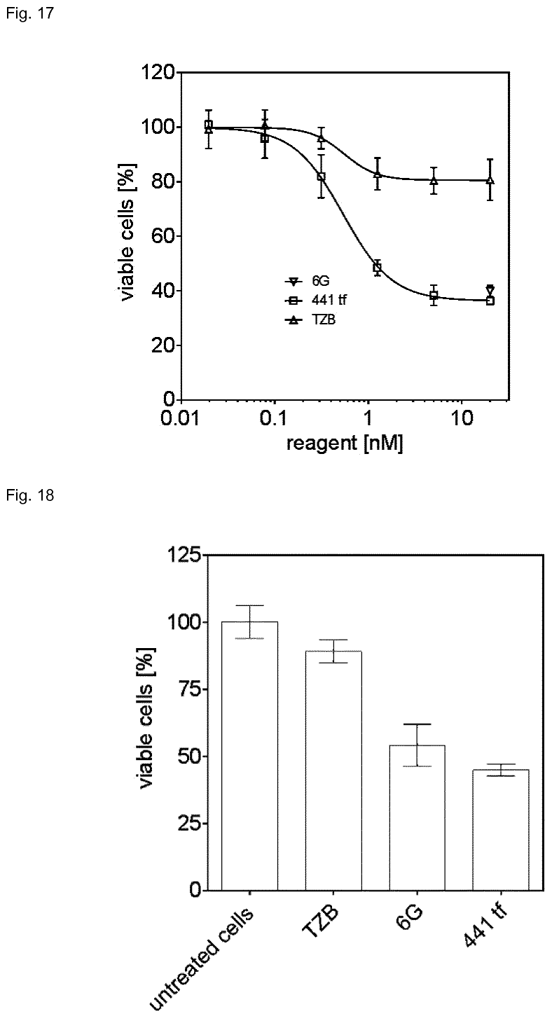

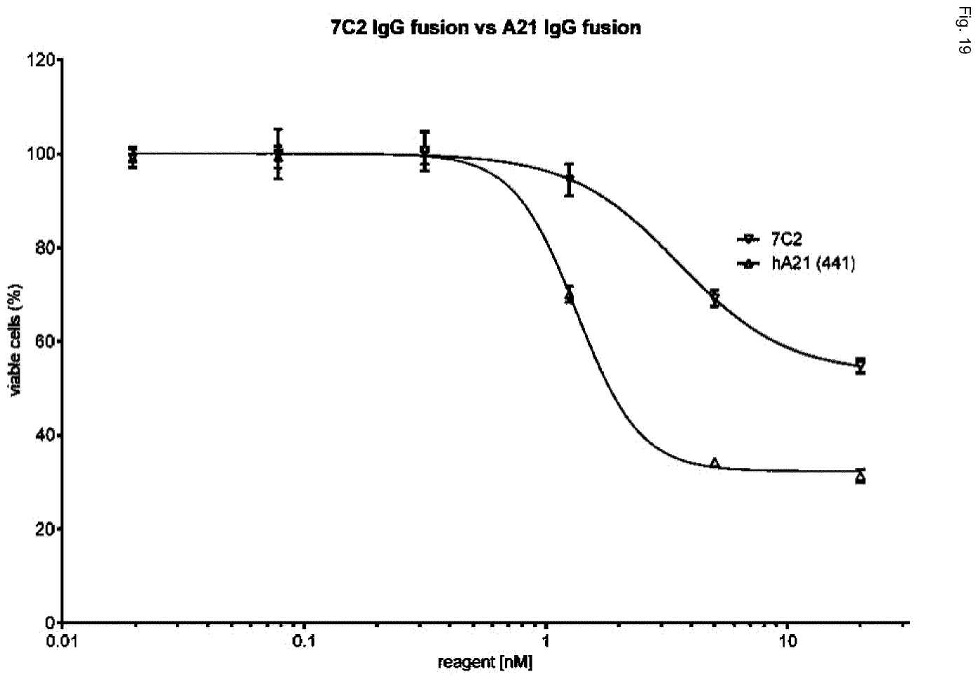

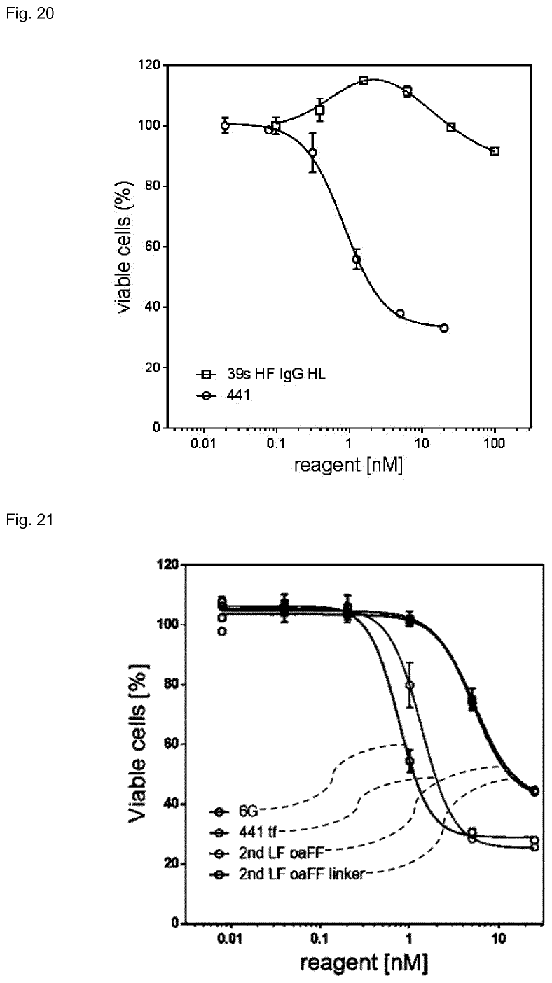

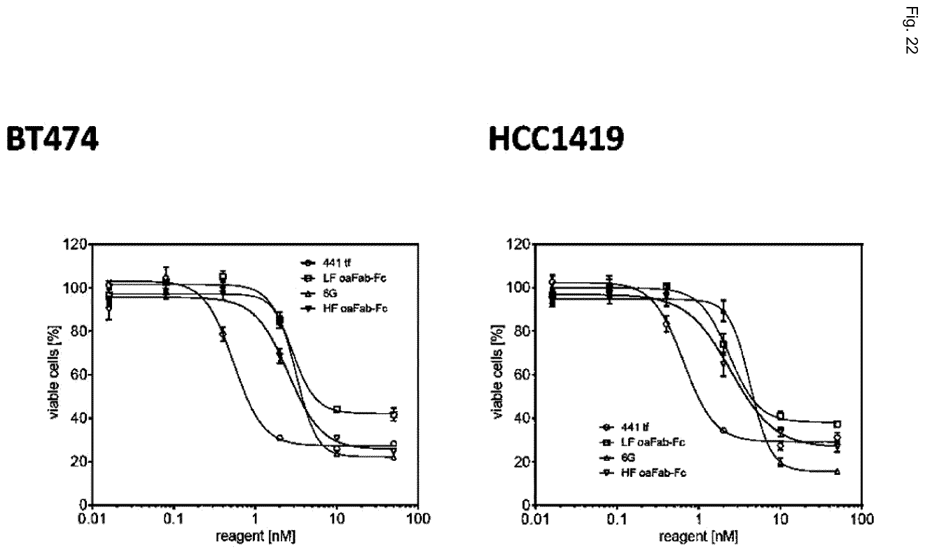

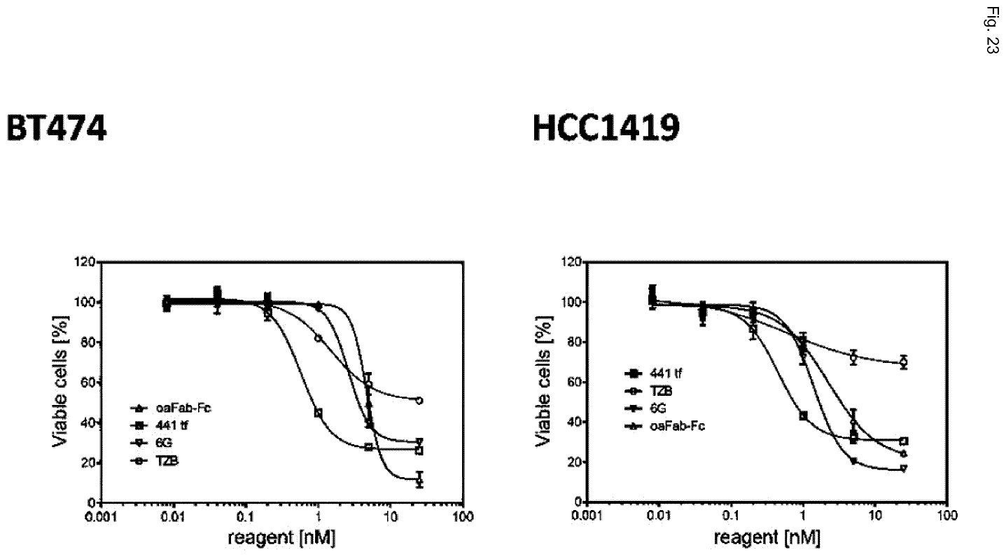

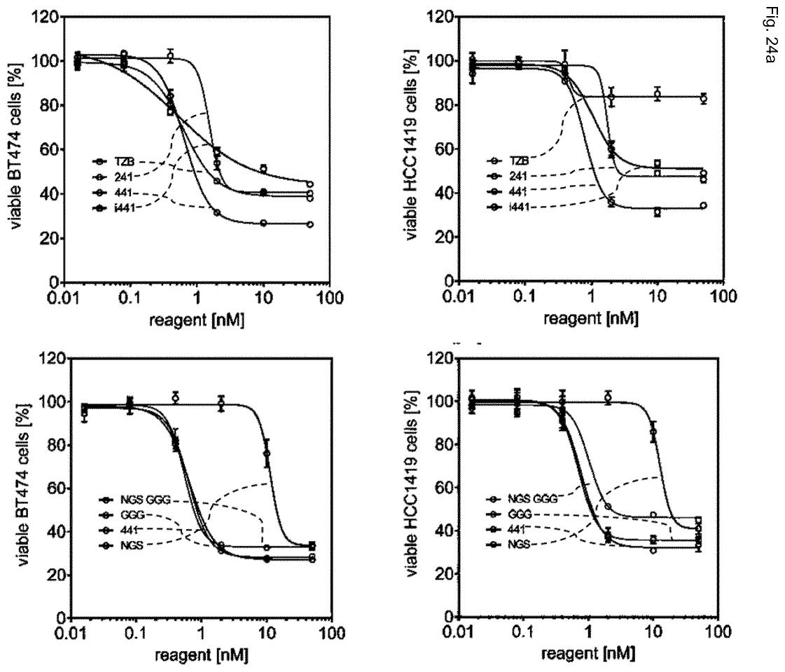

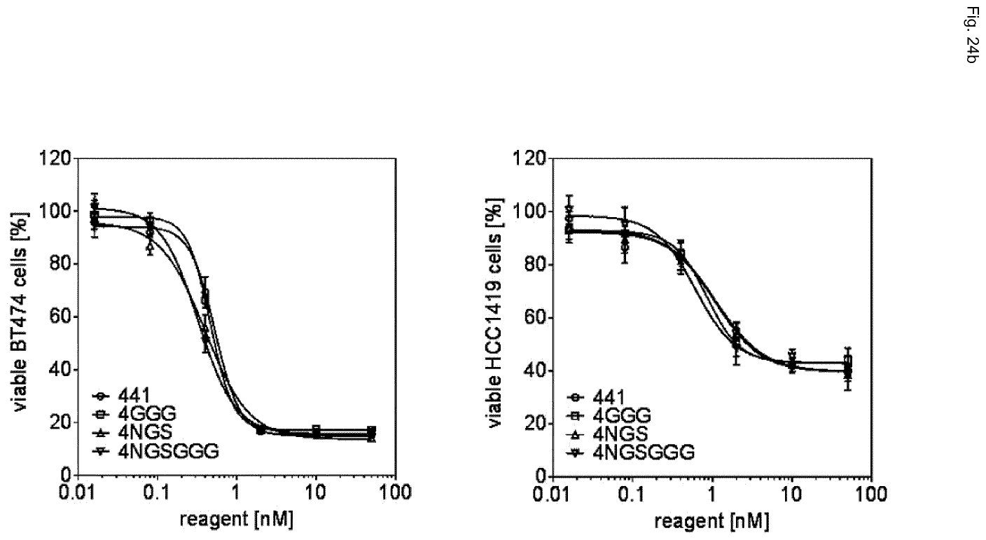

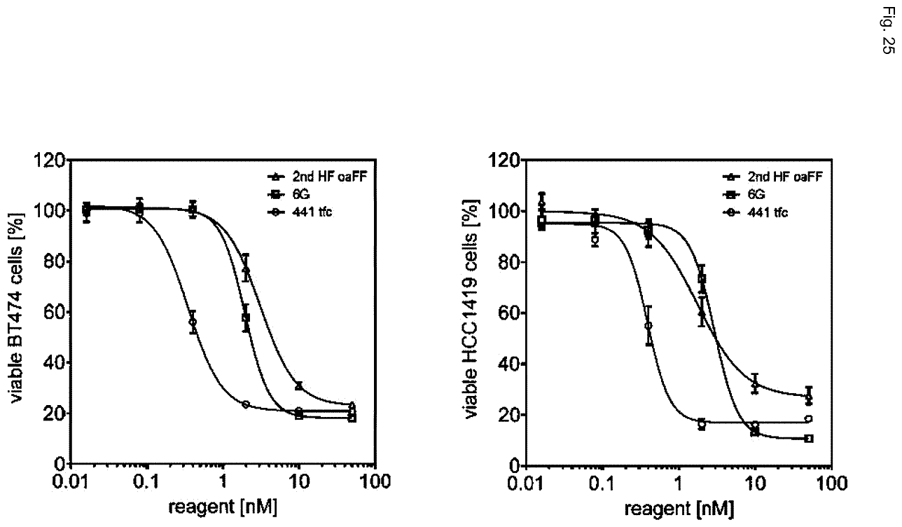

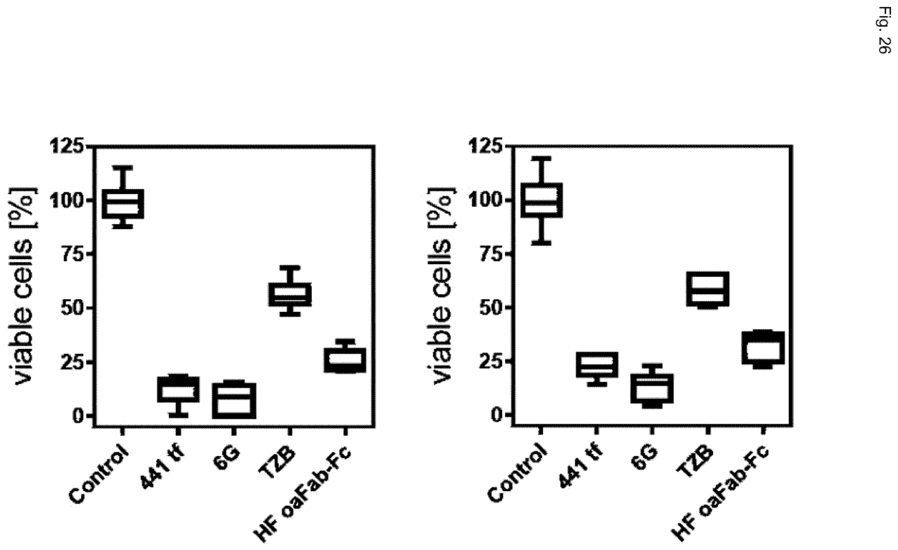

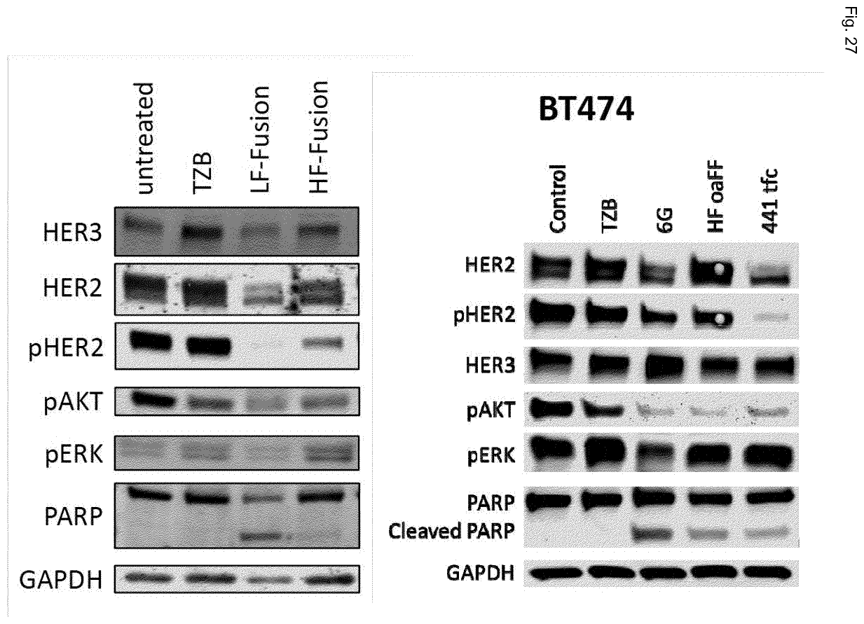

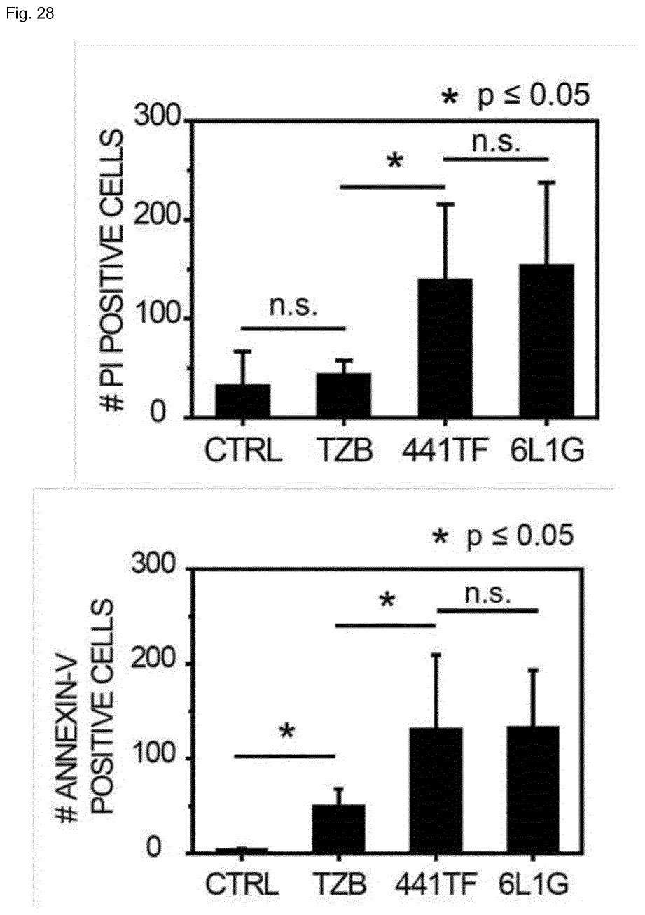

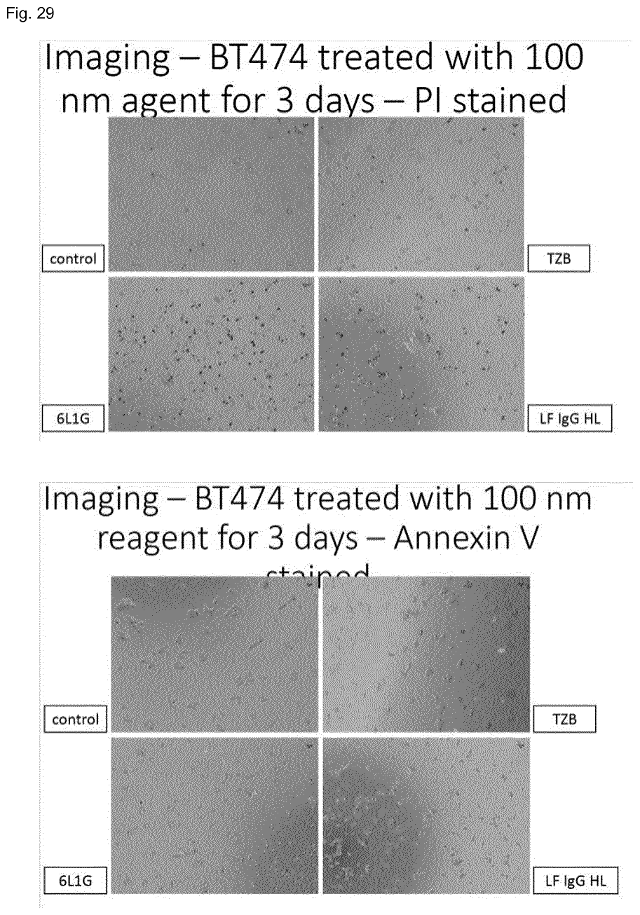

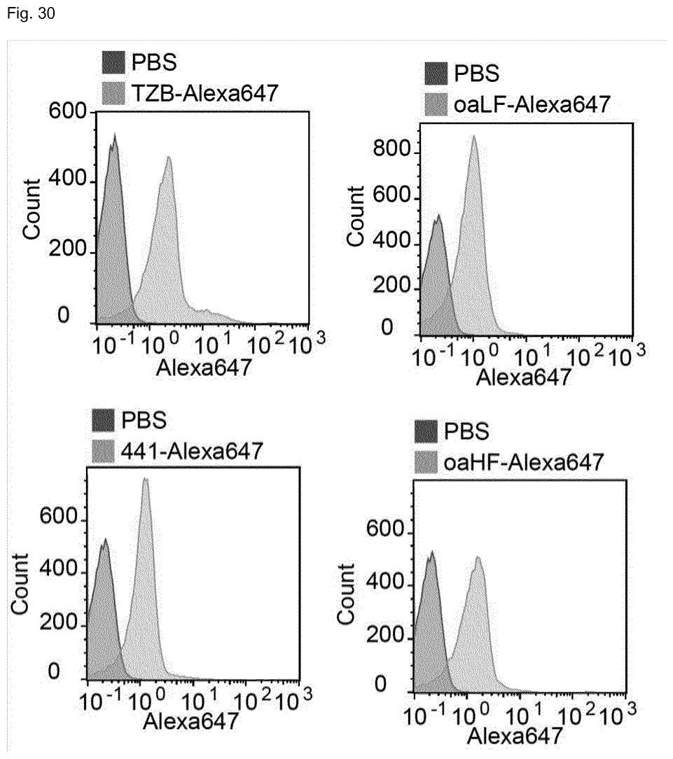

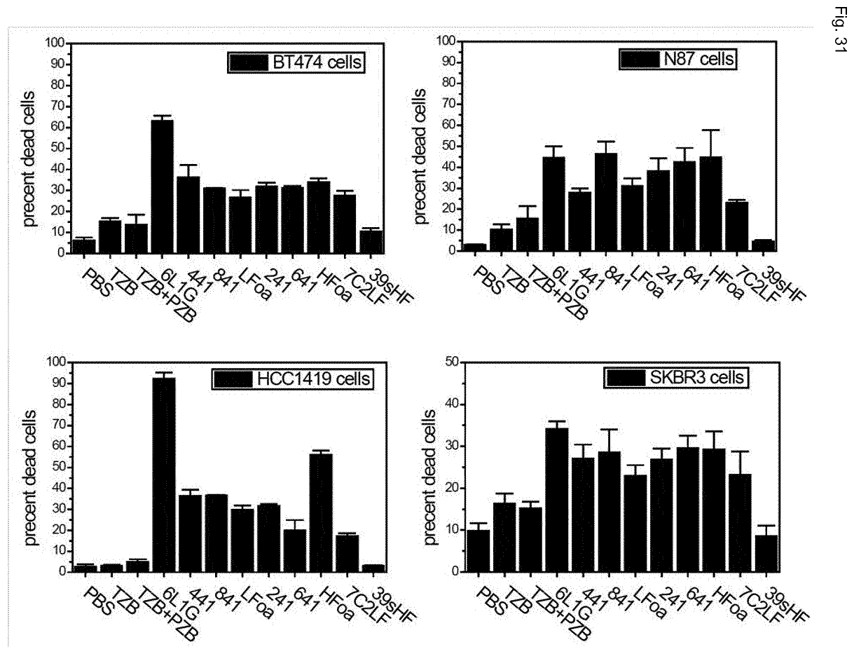

Data regarding preparation, and biological activity of the biparatopic IgG constructs are shown in FIGS. 3 to 26.

Protocol for Production of Biparatopic IgGs in CHOs Cells

Vector Design

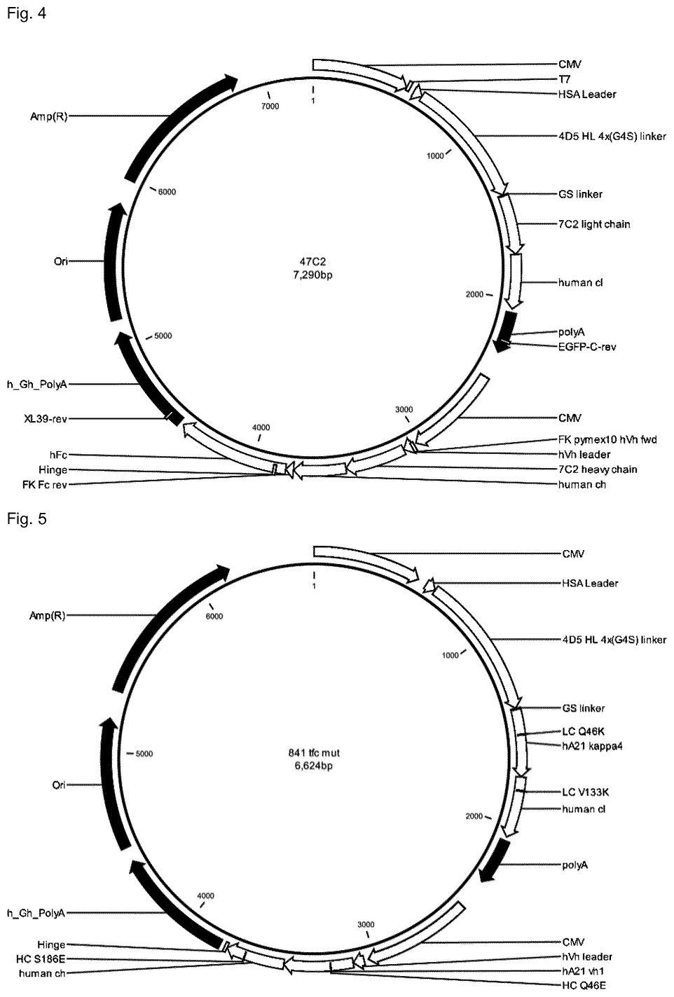

Bicistronic plasmids containing two expression cassettes or two vector systems were constructed for co-transfections. Derivatives of plasmid pYMex10 (Morphosys) were used for the bicistronic strategy. In the resulting plasmid constructs, the coding sequences of the polypeptide chains of the multimeric constructs were each under the control of an individual CMV promotor and terminated by a polyA tail signal as for example taken from bovine growth hormone or simian virus 40 (see FIGS. 3 and 4). pcDNA 3.1 (Thermo) derived vectors were used for co-transfections. Here, the individual polypeptide chains are on a separate plasmid reducing the risk of recombination of homologous elements and further allow to adjust molar ratios of plasmids for the transfections to improve the yield. Exchange of genes of interest is possible by standard cloning techniques. Examples of the resulting constructs are depicted in FIG. 5.

Expression in CHO-S

Exponentially growing CHO-S cells (Thermo) were seeded in CHOgro (Mirrus) at a density of four millions per ml in TPP600 bioreactors. Per ml of culture 3 pg of linear polyethylenimine (MW 25,000, PolySciences Inc) and 1.25 μg of highly pure plasmid DNA were added with in-between mixing. Eventually, cultures were supplemented with valproic acid to a final concentration of 1 mM. Proteins were expressed at 31° or 37° C., 8% CO2 and 180 rpm with a 50 mm throw in Kuhner ISF1-X shaker for up to 12 days.

Purification of Molecules

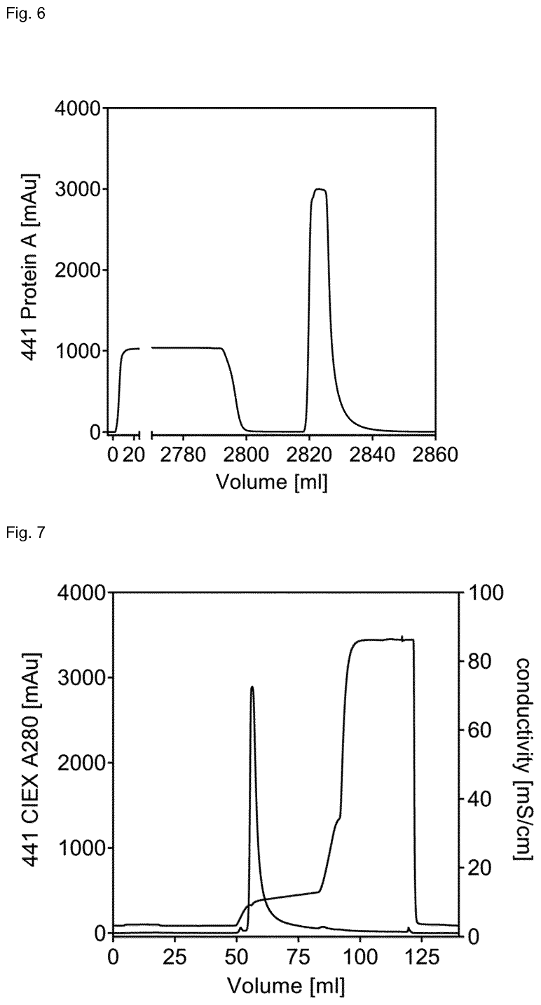

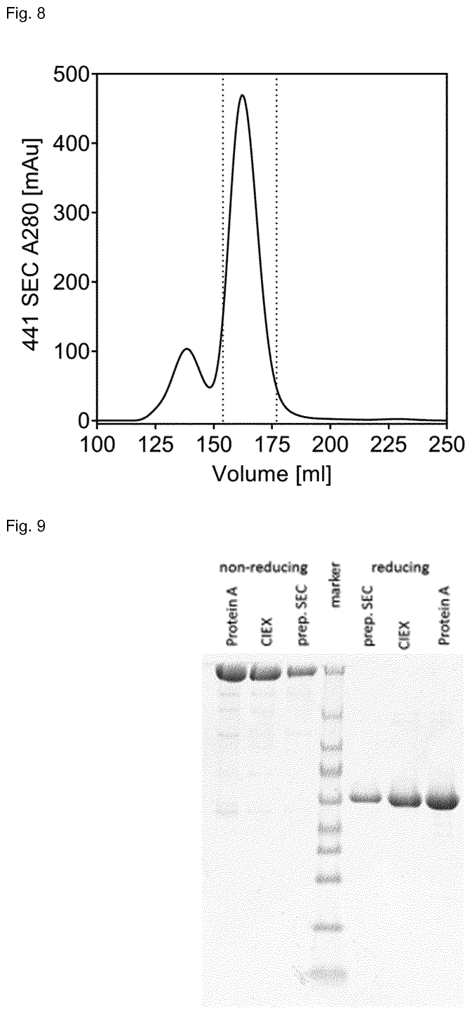

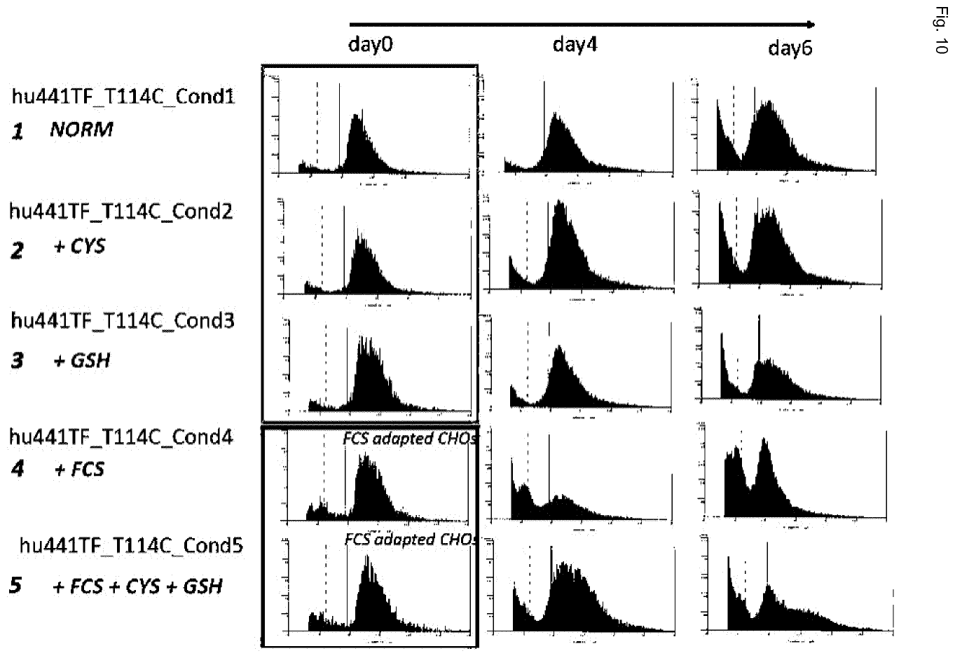

Expression cultures were harvested by centrifugation at 1400 rpm and 4° C. for 30 min. Supernatants were furthermore cleared by 0.22 μm filtration and adjusted to pH of 7. All subsequent purification steps were performed at 4° C. Supernatants were applied to PBS equilibrated rProtein A columns (GE) operated on a AEKTA Pure system. After PBS wash, bound protein was eluted by 0.1 M Glycine pH 2.75 (Chromatogram FIG. 6). Fractions of interest were pooled and buffer exchanged to 20 mM Bis/Tris methane 20 mM NaCI pH 6.75. Filtered protein solutions were loaded on Resource S (GE) columns and eluted with a gradient to 100% 20 mM Bis/Tris methane 1000 mM NaCI pH 6.75 (see FIG. 7). Desired fractions were pooled and if required polished on SEC (Superdex200 columns of adequate size; GE) for high purity (FIG. 8). Monomeric fractions were pooled, filtered, and purity analyzed on SDS page (FIG. 9). Eventually, pure protein was used for biochemical and cell assays as well as in-vivo studies.

HER2-Related Effects of Tetravalent Biparatopic IgGs

The tetrameric (tetravalent and biparatopic) polypeptide constructs 441 and 47C2 were tested in various assays, which are all well-known to the skilled person, and compared to the tetravalent IgG-fusion MED14276 (without a toxin), the dimeric bivalent biparatopic polypeptide constructs 841, 87C2 and Fc fusions thereof, the bivalent biparatopic DARPin construct 6L1G (see patent application WO 2014/060365 A1), single IgGs (TZB, PZB, A21 and 7C2) and combinations thereof.

Growth inhibition was tested in an XTT cell viability assay using live-cell high-content microscopy, Hoechst staining and cell count. As shown in Table 1, constructs 441, 47C2, 841, 87C2 and the Fc fusions of 841 and 87C2, 6L1G and the combination of TZB with A21 resulted in full growth inhibition, whereas single antibodies and TZB combined with PZB lead to partial inhibition. Surprisingly, in the absence of a cytotoxic drug, the antibody MED14276 stimulated growth of XTT cells.

The effect of the constructs on apoptosis/cell death were analyzed by live-cell high-content microscopy with annexin-V and PI staining or by detecting cleaved PARP in cell lysates by Western blot for analysis of PARP cleavage.

Constructs 441, 47C2, 841, 87C2 and their Fc fusions, 6L1G had an effect on apoptosis, whereas MED14276, single antibodies did not influence apoptosis, and TZB and A21 had a partial effect. The combination TZB+PZB is able to induce apoptosis in a very small fraction of cells or in fragile cell lines.

HER2 crosslinking on the cell surface, also termed “lockdown”, was measured by fluorescence recovery after photobleaching (FRAP) and single cell localization microscopy. A reduction of the FRAP signal indicates lower mobility of cells and therefore crosslinking in response to the polypeptide constructs.

441, 47C2 and 6L1G resulted in lockdown of receptors, and partial crosslinking effect was measured for 841, 87C2 and their Fc fusions as well as the antibody combinations TZB+PZB and TZB+A21. Single antibodies had no effect on crosslinking.

Furthermore, HER2 internalization into cells was analyzed by a surface protein internalization and degradation assay, confocal microscopy and flow cytometry as described in detail in example 2.

The tetravalent biparatopic constructs 441, 47C2 and MED14276 displayed a strong effect on HER2 internalization, whereas a recycling inhibition was detected for the combinations TZB+PZB and TZB+A21). The remaining constructs showed no effect.

HER2 degradation was tested by a surface protein internalization and degradation assay and Western blot detection of total HER2. Here, 441 and 47C2 lead to rapid strong degradation. MED14276 had an effect on degradation, but less strong compared to 441 and 47C2. In contrast, the antibody combinations resulted in slow degradation and the remaining constructs had no effect.

TABLE 1
Results of assays measuring HER2-related effects of polypeptide constructs

| | | | “Lockdown″ | | |
|---|---|---|---|---|---|
| | Growth | Apoptosis/ | (crosslinking | HER2 | HER2 |
| Construct | inhibition | cell death | on cells) | Internalization | Degradation |
| 441/47C2 | Yes | Yes | Yes | Strong | Rapid, |
| | | | | | Strong |
| MEDI4276 | No | No | Not | Same as 441 | Less than |
| (without | (stimulating!) | | determined but | | 441 |
| toxin) | | | expected | | |
| 841/87C2 | Yes | Yes | Partial | No | No |
| and Fc | | | | | |
| fusions | | | | | |
| thereof | | | | | |
| 6L1G | Yes | Yes | Yes | No | No |
| Single | Partial | No | No | No | No |
| antibodies | | | | | |
| TZB + PZB | Partial | No (yes) | Crosslinking | Recycling | Very slow |
| | | | | inhibition | |
| | | | | (very weak) | |
| TZB + A21 | Yes | Partial | Crosslinking | Recycling | Slow |
| | | | | inhibition | |

Table 2 shows the results of HER2 binding studies performed with the same constructs as the experiments described above. Binding was determined by flow cytometry and additionally by size-exclusion chromatography/multi angle light scattering (SEC-MALS) in case of complex formation between the biparatopic IgG constructs and HER2.

All constructs bound the extracellular domains 1, 2 and/or 4 as expected (see Table 2). 441, 47C2 and the combination TZB+A21 displayed an extremely slow off rate which was slower compared to the remaining constructs.

Interestingly, all biparatopic constructs resulted in a HER2 binding stoichiometry of close to 1:1. Single antibodies and antibody combinations only lead approximately to a 1:2 stoichiometry.

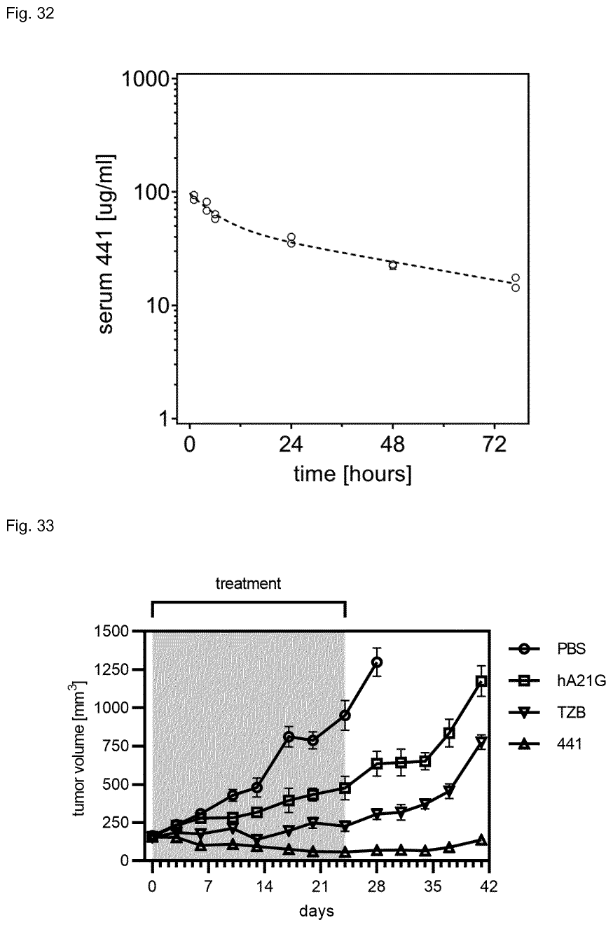

Serum half-life of construct 441 was further tested by intra venous injection in NSG mice and time resolved detection of biparatopic IgG in blood samples by ELISA. To determine the half-life of 441, 3 mg/kg of purified construct were intravenously injected in NSG mice. At indicated time points mice were blead, whole blood allowed to clot and sera gained by taking supernatants of centrifuged samples. A standard capture ELISA was used to evaluate serum levels of 441 (FIG. 32). Anti-human Fc antibody from mouse was directly coated on maxisorb plates. Bound 441 and sera spiked 441 standards were revealed by anti-kappa chain antibodies from goat conjugated to alkaline phosphatase. Data was fitted with a two-phase decay model and resulting half-life was calculated to be 4.3+45.3 hours (α and β phase).

TABLE 2
HER2 binding properties and other parameters of polypeptide constructs

| | | | Bound HER2 | Total # | | | Complex |
|---|---|---|---|---|---|---|---|
| | | | molecules | Paratope | | Molecular | size on |
| | | | on average | per | Serum Half | weight | cells (single |
| Construct | Binds to | Off Rate | in solution | molecule | life | kDa | molecule) |
| 441/47C2 | HER2 | extremely | Close to 1:1 | 4 | 45 hours | 200 | Large |
| | ECD1/4 | slow | | | | | |
| | | 2.7 × 10\hat{ } − 4 | | | | | |
| MEDI4276 | HER2 | Not | Close to 1:1 | 4 | rapid | 200 | Not |
| (without toxin) | ECD2/4 | determined | | | clearance | | determined |
| | | But expected | | | (app. 1-3 | | |
| | | extremely | | | day) | | |
| | | slow | | | | | |
| 841/87C2 and | HER2 | Very slow | Close to 1:1 | 2 | Not | 72(+FC = | Not |
| Fc fusions | ECD1/4 | | | | determined | 122) | determined |
| thereof | | | | | | | |
| 6L1G | HER2 | Very slow | Close to 1:1 | 2 | 5 min (12 | 32 | Medium |
| | ECD1/4 | | | | hours | | |
| | | | | | PEGylated) | | |
| Single | HER2 | Very slow | Close to 1:2 | 2 | 12 days | 145 | Small |
| antibodies | ECD1/2/4 | | | | | | |
| TZB + PZB | HER2 | Very slow | Close to 1:2 | 2 | 12 days | 145 | Not |
| | ECD2/4 | | | | | | determined |
| TZB + A21 | HER2 | extremely | Close to 1:2 | 2 | 12 days | 145 | Not |
| | ECD1/4 | slow | | | | | determined |
| | | 3.66 × 10\hat{ } − 4 | | | | | |

Surprisingly, the tetrameric tetravalent biparatopic constructs 441 and 47C2 lead to strong inhibition of cell proliferation, induced cell death by apoptosis and led to crosslinking of HER2 on the cell surface and induced strong HER2 internalization and strong HER2 degradation in addition to their excellent binding properties to HER2. 441 and 47C2 were superior to or scored equally well as all other constructs in all categories.

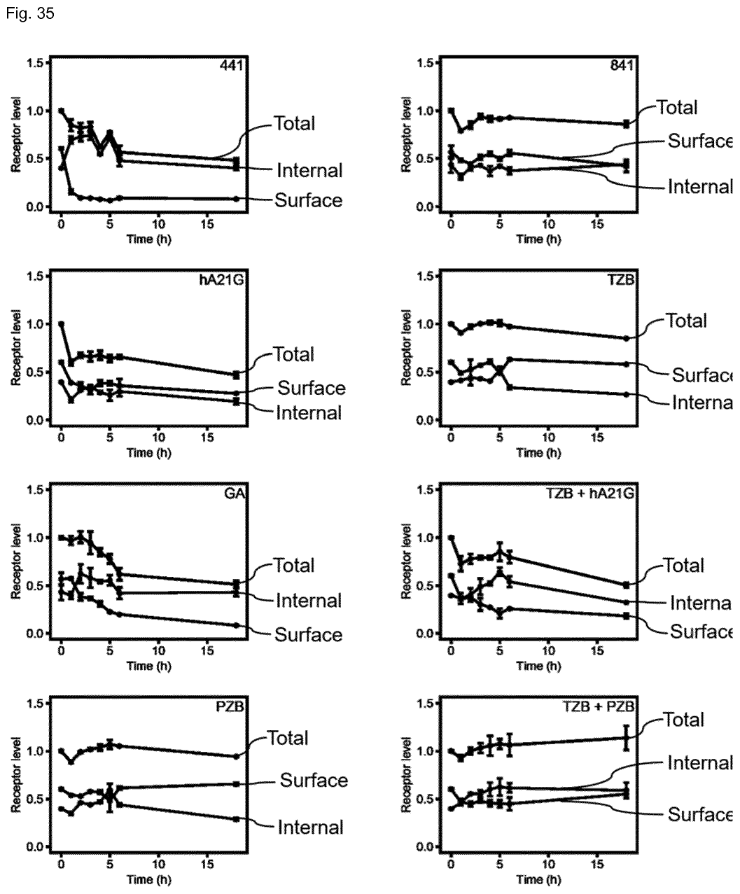

TZB+PZB and TZB+A21 resulted in a decrease of total HER2 (very weak in case of TZB+PZB) which was attributed to recycling inhibition, a mechanism by which HER2 is degraded without prior intracellular accumulation (see FIG. 35).

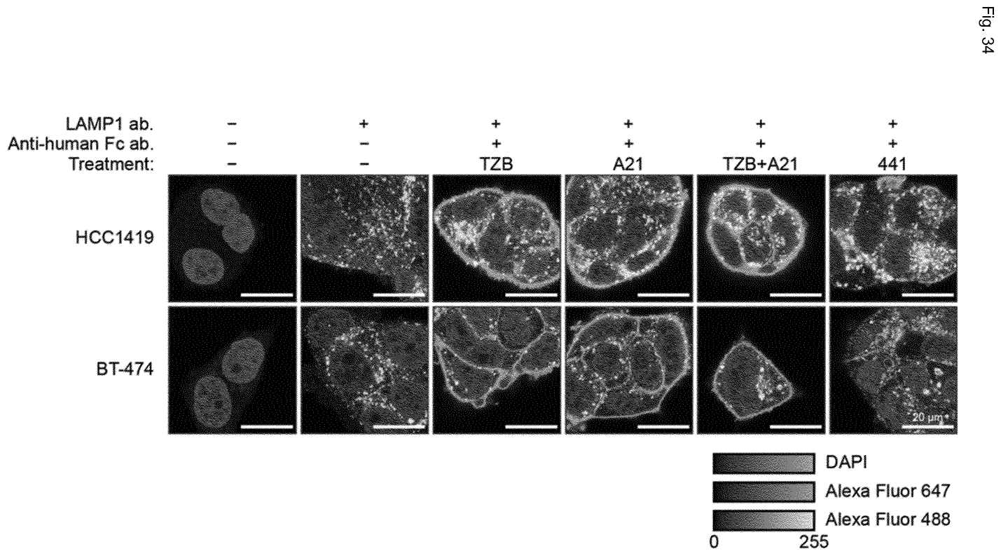

To determine the effect of construct 441 on tumor growth, SCID beige (Charles River) mice were inoculated on the right flank with five million N87 cells in 50% matrigel (Corning). After tumors had reached around 150 mm³ mice were treated with 10 mg/kg 441 for eight times with a three to four-day interval. Treated mice responded to 441 with tumor burden reduction. Growth arrest was initially seen for TZB (10 mg/kg) and hA21G (10 mg/kg) treated mice and tumors of control mice (labeled ‘PBS’ in FIG. 33) showed unhindered progression (FIG. 33).

### EXAMPLE 2

### Enhanced Internalization, Lysosomal Trafficking, and Degradation of HER2 by Revealed Molecule 441

Microscopy with BT-474 and HCC1419 breast cancer cells For microscopy of fixed samples, cells were seeded at a density of 4·10⁴ cm⁻² in p-slides

(Ibidi, cat. no. 80824) in complete medium. On the next day, cells were treated with the respective molecules. After 2 h, cells were once washed with Dulbecco's phosphate buffered saline (DPBS), and fixed by addition of 4% (w/v) paraformaldehyde dissolved in DPBS and incubation at room temperature for 10 min. Next, cells were washed twice with PBSBA+T (DPBS supplemented with 1% (w/v) bovine serum albumin (BSA), 0.1% (w/v) sodium azide, and 0.5% (w/v) Tween-20). Afterwards, cells were incubated in anti-LAMP antibody (Cell Signaling Technology, cat. no. D401S) dissolved at 1:150 (v/v) in PBSBA+T, further supplemented with 100 ng ml⁻¹ 2-(4-amidinophenyl)-1H-indo1-6-carboximidamide (DAPI) for 30 min at room temperature. Cells were then washed twice with PBSBA+T, and subsequently anti-mouse, conjugated to Alexa Fluor 488 (Thermo Fisher Scientific, cat. no. A11001) and anti-human, conjugated to Alexa Fluor 647 (Thermo Fisher Scientific, cat. no. A-21445) antibodies from goat, dissolved in PBSBA+T, were added and incubated for 30 min at room temperature. Next, cells were washed twice with PBSBA+T, fixed once more by addition of 4% (w/v) paraformaldehyde dissolved in DPBS and incubation at room temperature for 10 min, finally washed once with PBSA, and stored in PBSA (DPBS supplemented with 0.1% (w/v) sodium azide) at 4 ° C. until measurement. Imaging was performed on a SP5 confocal laser scanning microscope (Leica). The images show that for both cell lines, only 441 was able to induce its rapid internalization, and shows strong colocalization with lysosomal (LAMP1-positive) compartments.

Surface Protein Internalization and Degradation Assay

To quantify internalization and degradation of HER2 upon treatments, we performed a quantitative surface protein internalization and degradation assay was performed. In brief, a stable Flp-In TREx HEK293 cell line (Thermo Fisher Scientific, cat. no. K650001) was generated according to the instructions of the manufacturer, in which a HaloTag-HER2 receptor fusion can be overexpressed upon induction. For the assay, cells were seeded two days before the first treatment, and one day before treatment, doxycycline was added to induce stable overexpression for 24 h. Treatments (100 nM) were added at indicated time points, referring to the time of cell labeling.

After completion of the treatment time intervals, cells were labeled in a two-step procedure. A HaloTag ligand containing to Alexa Fluor 660 (HTL-AF660, Promega, cat. no. G8472), which is completely cell-impermeable and therefore stains surface receptors only, was coupled in a first labeling step. A cell-permeable HaloTag ligand containing tetramethyl rhodamine (HTL-TMR, Promega, cat. no. G8252), was, in the second step, applied to stain all receptor fusion, which resides in intracellular compartments. Thus, signals originating from surface and internal receptor are detected in separate channels on a flow cytometer. Therefore, information regarding the localization, and using the rescaling procedure described below, about the quantitative distribution, can be obtained. A commercially available dead-cell stain was used for exclusion of permeabilized (dead) cells from analysis, for which all receptor would appear to be on the surface. Fluorescence intensities in each channel for 2′000-10′000 cells was recorded using a LSR II Fortessa (BD), and single, non-permeabilized cells gated. Mean fluorescence intensities of these populations were obtained using FlowJo 10.4 (FlowJo).

Data Processing

To correct for different detection efficiencies of the flow cytometry instrument in the channels for AF660 and TMR, the data were scaled, yielding relative abundances. A control sample (utr.,s.), in which the first (HTL-AF660-labeling) step is omitted, is required to this end. In this sample, all HaloTag molecules will react with HTL-TMR in this “single” (s.) labeling procedure, irrespective of their localization. Using the mean fluorescence intensities (MFI) of the singlet, non-permeabilized cell population, the normalized (feature-scaled) signal S_{TMR} in the TMR channel for a samples is obtained by normalizing to a single-labeled, untreated control sample (utr.,s.) and background subtraction:

$${S}_{TMR} ( sample ) = \frac{{MFI}_{TMR} ( sample ) - {MFI}_{TMR} ( utr . , unlab . )}{{MFI}_{TMR} ( utr . , s . ) - {MFI}_{TMR} ( utr . , unlab . )} , \qquad \# ( eq . S1 )$$

where utr.,unlab. represents an untreated, unlabeled control (showing only autofluorescence).

The first, surface-labeling step with cell-impermeable dye is virtually saturating in the complete two-step (double) labeling procedure. The normalized surface signal S_{AF660} can thus be defined in:

$${S}_{AF 660} ( sample ) = \frac{{MFI}_{AF 660} ( sample ) - {MFI}_{AF 660} ( utr . , unlab . )}{{MFI}_{AF 660} ( utr . , s . ) - {MFI}_{Af 660} ( utr . , unlab . )} . \qquad \# ( eq . S2 )$$

The signal of a sample can be related to the surface signal from a double-labeled control (utr.,d.) and does not require a separate single-stained sample, however, because internal receptor is not accessible to HTL-AF660 and thus cannot be stained.

ΔS_{TMR}, the difference from the single to the double labeling procedure in the TMR channel, now exactly corresponds to the number of molecules, which were blocked by the first, surface-specific step. The signal in the AF660 channel in the same double labeling experiment also is a direct correlate of this number of molecules:

ΔS_{TMR}=S_{TMR}(utr.,s.)−S_{TMR}(utr.,d.)=S_{AF660,scaled}(utr.,d.)# (eq. S3).

A correction factor C_{A} can thus be defined, which relates the measured intensity S_{AF660}(utr.,d.) (recorded in the AF660 channel) to S_{AF660,scaled}(utr.,d.) (in the scale of the TMR channel):

S_{AF660,scaled}(Utr.,d.)=S_{AF660}(utr.,d.)×C_{A}# (eq. S4).

Using signals from single-labeled and double-labeled, untreated cells, calculation of C_{A} is possible as follows:

$${C}_{A} = \frac{{S}_{TMR} ( utr . , s . ) - {S}_{TMR} ( utr . , d . )}{{S}_{AF 660} ( utr . , d . )} = \frac{100 \% - {S}_{TMR} ( utr . , d . )}{{S}_{AF 660} ( utr . , d . )} , \qquad \# ( eq . S5 )$$

taking into consideration that the TMR signal of single-labeled, untreated cells was, using eq. S1, scaled to 100% before. Correction of the signals recorded in the AF660 channel, can, for treated samples, now be done according to:

S_{AF660,scaled}(tre,d.)=C_{A}×S_{AF660}(tre.,d.)# (eq. S6)

S_{TMR}(tre.,d.) and S_{AF660,scaled}(tre.,d.) then truly represent the abundance of internal and surface protein, respectively, and the sum S_{AF660,scaled}(tre.,d.)+S_{TMR}(tre.,d.) represents the amount of total protein, for a double-labeled, treated sample, always relative to an untreated control sample.

Data scaling as described above was performed and the results plotting using MATLAB R2017b (MathWorks), R 3.5.1, Prism 6.07 (GraphPad), and Excel2016 (Microsoft). The results are shown in FIG. 35. In contrast to all other constructs, construct 441 showed almost HER2 internalization after less than 5 minutes.

1. A tetrameric polypeptide comprising or consisting of
a. a first polypeptide chain comprising a first VL antigen binding domain and a first CL constant domain,
b. a second polypeptide chain comprising a first VH antigen binding domain, a first CH1 constant domain, a first CH2 constant domain and a first CH3 constant domain,
c. a first ligand that specifically binds to a HER2 D4 epitope, wherein said first ligand is comprised in said first polypeptide chain and linked to the N-terminus of said first VL antigen binding domain by a first interdomain amino acid linker, or said first ligand is comprised in said second polypeptide chain and linked to the N-terminus of said first VH antigen binding domain by a first interdomain amino acid linker,
d. wherein the first VL antigen binding domain of the first polypeptide chain and the first VH antigen binding domain of the second polypeptide chain together constitute a second ligand, particularly a Fab domain, that specifically binds to a HER2 D1 epitope,
e. a third polypeptide chain comprising a second VL antigen binding domain and a second CL constant domain,
f. a fourth polypeptide chain comprising a second VH antigen binding domain, a second CH1 constant domain, a second CH2 constant domain and a second CH3 constant domain,
g. a third ligand that specifically binds to a HER2 D4 epitope, wherein said third ligand is comprised in said third polypeptide chain and linked to the N-terminus of said second VL antigen binding domain by a second interdomain amino acid linker, or said third ligand is comprised in said fourth polypeptide chain and linked to the N-terminus of said second VH antigen binding domain by a second interdomain amino acid linker,
h. wherein the second VL antigen binding domain of the third polypeptide chain and the second VH antigen binding domain of the fourth polypeptide chain together constitute a fourth ligand, particularly an Fab domain, that specifically binds to a HER2 D1 epitope.

2. The polypeptide according to claim 1, wherein
said first polypeptide chain and said third polypeptide chain and/or said second polypeptide chain and said fourth polypeptide chain are characterized by a sequence identity with each other of 70% or more, particularly 80% or more, more particularly 90% or more, even more particularly 95% or more, wherein most particularly said first polypeptide chain and said third polypeptide chain and/or said second polypeptide chain and said fourth polypeptide chain are identical and/or wherein
said first ligand and said third ligand are characterized by a sequence identity with each other of 70% or more, particularly 80% or more, more particularly 90% or more, even more particularly 95% or more, wherein most particularly said first ligand and said third ligand are identical.

3. The tetrameric polypeptide according to any one of the preceding claims, wherein said first CH2 constant domain and said first CH3 constant domain of said second polypeptide chain interact with, particularly are covalently linked with, said second CH2 constant domain and said second CH3 constant domain of said fourth polypeptide chain, such that a tetrameric polypeptide is formed.

4. The tetrameric polypeptide according to any one of the preceding claims, wherein said second polypeptide chain comprises a first hinge region between said first CH1 constant domain and said first CH2 constant domain, and said fourth polypeptide chain comprises a second hinge region between said second CH1 constant domain and said second CH2 constant domain, wherein said first hinge region and said second hinge region mediate complex formation between said second polypeptide chain and said fourth polypeptide chain, particularly by at least one disulphide bond, such that a tetrameric polypeptide is formed.

5. The polypeptide according to any one of the preceding claims, wherein said first ligand and/or said third ligand comprises or consists of a single-chain variable fragment polypeptide chain comprising an scFv heavy chain, an scFv linker chain, and an scFv light chain, wherein particularly
a. said scFv heavy chain of said first ligand and/or said third ligand comprises a peptide sequence characterized by a sequence identity of 70% or more, more particularly 80% or more, even more particularly 90% or more, even more particularly 95% or more, with a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, wherein most particularly said scFv heavy chain of said first ligand and/or said third ligand comprises a peptide sequence identical to a peptide sequence selected from SEQ ID No. 15, SEQ ID No. 21, SEQ ID No. 22, SEQ ID No. 23, SEQ ID No. 44, SEQ ID No. 53, SEQ ID No. 54 and SEQ ID No. 80, and b. said scFv light chain of said first ligand and/or said third ligand comprises a peptide sequence characterized by a sequence identity of 70% or more, more particularly 80% or more, even more particularly 90% or more, even more particularly 95% or more, with a peptide sequence selected from SEQ ID No. 14, SEQ ID No. 24, SEQ ID No. 25, SEQ ID No. 26, SEQ ID No. 43 and SEQ ID No. 81, wherein most particularly said scFv light chain of said first ligand and/or said third ligand comprises a peptide sequence identical to a peptide sequence selected from SEQ ID No. 14, SEQ ID No. 24, SEQ ID No. 25, SEQ ID No. 26, SEQ ID No. 43 and SEQ ID No. 81.

6. The polypeptide according to claim 5, wherein said scFv linker chain comprises a peptide sequence characterized by a sequence identity of 70% or more, particularly 80% or more, more particularly 90% or more, even more particularly 95% or more, with SEQ ID No. 16, wherein most particularly said scFv linker chain comprises a peptide sequence identical to SEQ ID No. 16.

7. The polypeptide according to any one of the preceding claims, wherein
a. said first polypeptide chain and/or said third polypeptide chain comprises a peptide sequence characterized by a sequence identity of 70% or more, particularly 80% or more, more particularly 90% or more, even more particularly 95% or more, with a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID 76, wherein most particularly said first polypeptide chain and/or said third polypeptide chain comprises a peptide sequence identical to a peptide sequence selected from SEQ ID No. 18, SEQ ID No. 30, SEQ ID No. 31, SEQ ID No. 32, SEQ ID No. 39, SEQ ID No. 41, SEQ ID No. 50 and SEQ ID No. 76, and
b. said second polypeptide chain and/or said fourth polypeptide chain comprises a peptide sequence characterized by a sequence identity of 70% or more, particularly 80% or more, more particularly 90% or more, even more particularly 95% or more, with a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77, wherein most particularly said second polypeptide chain and/or said fourth polypeptide chain comprises a peptide sequence identical to a peptide sequence selected from SEQ ID No. 19, SEQ ID No. 20, SEQ ID No. 27, SEQ ID No. 28, SEQ ID No. 29, SEQ ID No. 40, SEQ ID No. 42, SEQ ID No. 51, SEQ ID No. 52 and SEQ ID 77.

8. The polypeptide according to any one of the preceding claims, wherein
a. said first polypeptide chain and/or said third polypeptide chain comprises a peptide sequence characterized by a sequence identity of 70% or more, particularly 80% or more, more particularly 90% or more, even more particularly 95% or more, with SEQ ID No. 36, SEQ ID No. 37, SEQ ID No. 38 and SEQ ID No. 78, wherein most particularly said first polypeptide chain and/or said third polypeptide chain comprises a peptide sequence identical to SEQ ID No. 36, SEQ ID No. 37, SEQ ID No. 38 and SEQ ID No. 78, and
b. said second polypeptide chain and/or said fourth polypeptide chain comprises a peptide sequence characterized by a sequence identity of 70% or more, particularly 80% or more, more particularly 90% or more, even more particularly 95% or more, with SEQ ID No. 33, SEQ ID No. 34, SEQ ID No. 35 and SEQ ID No. 79, wherein most particularly said second polypeptide chain and/or said fourth polypeptide chain comprises a peptide sequence identical to SEQ ID No. 33, SEQ ID No. 34, SEQ ID No. 35 and SEQ ID No. 79.

9. The polypeptide according to any one of the preceding claims, wherein
a. said first polypeptide chain and/or said third polypeptide chain is characterized by a sequence identity of 70% or more, particularly 80% or more, more particularly 90% or more, even more particularly 95% or more, with SEQ ID No. 1, wherein most particularly said first polypeptide chain and/or said third polypeptide chain is identical to SEQ ID No. 1, and
b. said second polypeptide chain and/or said fourth polypeptide chain is characterized by a sequence identity of 70% or more, particularly 80% or more, more particularly 90% or more, even more particularly 95% or more, with SEQ ID No. 2, wherein most particularly said second polypeptide chain and/or said fourth polypeptide chain is identical to SEQ ID No. 2.

10. The polypeptide according to any one of the preceding claims, wherein
a. said first polypeptide chain and/or said third polypeptide chain is characterized by a sequence identity of 70% or more, particularly 80% or more, more particularly 90% or more, even more particularly 95% or more, with SEQ ID No. 3, wherein most particularly said first polypeptide chain and/or said third polypeptide chain is identical to SEQ ID No. 3, and
b. said second polypeptide chain and/or said fourth polypeptide chain is characterized by a sequence identity of 70% or more, particularly 80% or more, more particularly 90% or more, even more particularly 95% or more, with SEQ ID No. 4, wherein most particularly said second polypeptide chain and/or said fourth polypeptide chain is identical to SEQ ID No. 4.

11. The polypeptide according to any one of the preceding claims, wherein said first interdomain amino acid linker and/or said second interdomain amino acid linker consists of 1, 2, 3, 4, 5, 6, 7, 8, 9, 10, 11, 12, 13, 14, 15, 16, 17, 18, 19 or 20 amino acids, and wherein said first interdomain amino acid linker and/or said second interdomain amino acid linker comprises or consists of amino acids G, A, J, S, T, P, C, V, M and E, particularly wherein said first interdomain amino acid linker and/or said second interdomain amino acid linker comprises or consists of amino acids G, S, A and T, more particularly wherein said first interdomain amino acid linker and/or said second interdomain amino acid linker is characterized by an amino acid sequence (GGGGS)n, with n being 1, 2, 3, 4 or 5 or said first interdomain amino acid linker and/or said second interdomain amino acid linker comprises or consists of a peptide sequence selected from one of SEQ ID No. 17, SEQ ID 55 to SQ ID 69 and SEQ ID 82 to SEQ ID 91 or a functional equivalent peptide sequence characterized by a sequence identity of at least 70%.

12. The polypeptide according to any one of the claims 1 to 11 for use in a method for the prevention or treatment of a malignant neoplastic disease associated with expression of HER2.

13. An isolated nucleic acid encoding at least one of the first polypeptide chain, the second polypeptide chain, the third polypeptide chain and the fourth polypeptide chain of the polypeptide according to any one of the claims 1 to 11.

14. A host cell which is adapted to produce at least one of the first polypeptide chain, the second polypeptide chain, the third polypeptide chain and the fourth polypeptide chain of the polypeptide according to any one of the claims 1 to 11, wherein particularly the host cell comprises the isolated nucleic acid according to claim 13, such that the host cell is able to produce at least one of the first polypeptide chain, the second polypeptide chain, the third polypeptide chain and the fourth polypeptide chain of the polypeptide according to any one of the claims 1 to 11.

15. A method for obtaining the polypeptide according to any one of the claims 1 to 11, wherein the method comprises culturing the host cell according to claim 14, so that at least one of the first polypeptide chain, the second polypeptide chain, the third polypeptide chain and the fourth polypeptide chain of the polypeptide according to any one of the claims 1 to 11 is produced.


SEQUENCE LISTING (93 sequences)

SEQ ID NO: 1 - protein, 469 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Artificial Sequence
  Antibody fusion construct
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 3 - protein, 468 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Antibody fusion construct
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 5 - protein, 223 aa, Artificial Sequence
  Antibody fusion construct
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 6 - protein, 221 aa, Artificial Sequence
  Antibody fusion construct
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 7 - protein, 696 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 8 - protein, 450 aa, Artificial Sequence
  Antibody fusion construct
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 9 - protein, 695 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 10 - protein, 448 aa, Artificial Sequence
  Antibody fusion construct
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 11 - protein, 220 aa, Artificial Sequence
  Antibody fusion construct
DIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 697 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 13 - protein, 472 aa, Artificial Sequence
  Antibody fusion construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 14 - protein, 107 aa, Artificial Sequence [sequence omitted]

SEQ ID NO: 15 - protein, 120 aa, Artificial Sequence [sequence omitted]

SEQ ID NO: 16 - protein, 20 aa, Artificial Sequence
  scFv linker
GGGGSGGGGSGGGGSGGGGS

SEQ ID NO: 17 - protein, 2 aa, Artificial Sequence
  linker
GS

SEQ ID NO: 18 - protein, 220 aa, Artificial Sequence; identical to SEQ ID NO: 11
  Antibody light chain

SEQ ID NO: 19 - protein, 448 aa, Artificial Sequence; identical to SEQ ID NO: 2
  Antibody heavy chain

SEQ ID NO: 20 - protein, 223 aa, Artificial Sequence; identical to SEQ ID NO: 5
  Antibody heavy chain

SEQ ID NO: 21 - protein, 10 aa, Artificial Sequence
  Antibody fragment
GFNIKDTYIH

SEQ ID NO: 22 - protein, 17 aa, Artificial Sequence
  Antibody fragment
RIYPTNGYTRYADSVKG

SEQ ID NO: 23 - protein, 11 aa, Artificial Sequence
  Antibody fragment
WGGDGFYAMDY

SEQ ID NO: 24 - protein, 11 aa, Artificial Sequence
  Antibody fragment
RASQDVNTAVA

SEQ ID NO: 25 - protein, 7 aa, Artificial Sequence
  Antibody fragment
SASFLYS

SEQ ID NO: 26 - protein, 9 aa, Artificial Sequence
  Antibody fragment
QQHYTTPPT

SEQ ID NO: 27 - protein, 10 aa, Artificial Sequence
  Antibody fragment
GYSFTGYFIN

SEQ ID NO: 28 - protein, 17 aa, Artificial Sequence
  Antibody fragment
HISSSYATSTYNQKFQG

SEQ ID NO: 29 - protein, 11 aa, Artificial Sequence
  Antibody fragment
SGNYEEYAMDY

SEQ ID NO: 30 - protein, 17 aa, Artificial Sequence
  Antibody construct
RSSQTLLYSNNQKNYLA

SEQ ID NO: 31 - protein, 7 aa, Artificial Sequence
  Antibody fragment
WAFTRKS

SEQ ID NO: 32 - protein, 9 aa, Artificial Sequence
  Antibody fragment
QQYSNYPWT

SEQ ID NO: 33 - protein, 10 aa, Artificial Sequence
  Antibody fragment
GYSFTGYWMN

SEQ ID NO: 34 - protein, 16 aa, Artificial Sequence
  Antibody fragment
MIHPLDAEIRANQKFR

SEQ ID NO: 35 - protein, 9 aa, Artificial Sequence
  Antibody fragment
GTYDGGFEY

SEQ ID NO: 36 - protein, 15 aa, Artificial Sequence
  Antibody fragment
RASQSVSGSRFTYMH

SEQ ID NO: 37 - protein, 7 aa, Artificial Sequence
  Antibody fragment
YASILES

SEQ ID NO: 38 - protein, 10 aa, Artificial Sequence
  Antibody fragment
QHSWEIPPWT

SEQ ID NO: 39 - protein, 220 aa, Artificial Sequence
  Antibody light chain
DIVLTQSPDSLAVSLGERATINCRSSQTLLYSNNQKNYLAWYQQKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 223 aa, Artificial Sequence
  Antibody heavy chain
QVQLVQSGAEVKKPGASVKVSCKASGYSFTGYFINWVRQAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 41 - protein, 220 aa, Artificial Sequence
  Antibody light chain
DIVLTQSPDSLAVSLGERATINCRSSQPLEYSNNQWNYLAWYQKKPGQPPKLLISWAFTRKSGVPDRFSGSGSGTDFTLTISSLQAEDVAVYYCGQYSDYPNTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 448 aa, Artificial Sequence
  Antibody heavy chain
QVQLVQSGAEVKKPGASVKVSCKASGYPFTQYFIHWVREAPGQGLEWMGHISSSYATVDYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 43 - protein, 107 aa, Artificial Sequence
  Antibody light chain
DIQMTQSPSSLSASVGDRVTITCRASQDVSTAVAWYQQKPGKAPKLLIYSASFLYSGVPSRFSGSRSGTDFTLTISSLQPEDFATYYCQQHYTTPPTFGQGTKVEIK

SEQ ID NO: 44 - protein, 120 aa, Artificial Sequence
  Antibody heavy chain
EVQLVESGGGLVQPGGSLRLSCAASGFNIKDTYIHWVRQAPGKGLEWVARIYPTNAYTRYADSVKGRFTISADTSKNTAYLQMNSLRAEDTAVYYCSRWGGTGFYAMDYWGQGTLVTVSS

SEQ ID NO: 45 - protein, 217 aa, Artificial Sequence
  Antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 217 aa, Artificial Sequence
  Antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, Artificial Sequence
  Antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 48 - protein, 217 aa, Artificial Sequence
  Antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 217 aa, Artificial Sequence
  Antibody fragment
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 220 aa, Artificial Sequence
  Antibody light chain
DIQLTQSPSSLSASVGDRVTITCRSSQTLLYSNNQKNYLAWYQQKPGKAPKLLISWAFTRKSGVPSRFSGSGSGTDFTLTISSLQPEDFATYYCQQYSNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 223 aa, Artificial Sequence
  Antibody heavy chain
EVQLVQSGPELVQPGGSVRISCAASGYSFTGYFINWVKQAPGKGLEWISHISSSYATSTYNQSFKGRATFSVDTSSSTAYMQLNSLRAEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 52 - protein, 222 aa, Artificial Sequence
  Antibody heavy chain
VQLVESGGGLVQPGGSLRLSCAASGYSFTGYFINWVRQAPGKGLEWVSHISSSYATSTYNQSVKGRFTFSVDTSSSTAYLQMNSLRAEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 53 - protein, 120 aa, Artificial Sequence
  Antibody heavy chain
QVQLVQSGAEVKKPGASVKVSCKASGFNIKDTYIHWVRQAPGQGLEQMGRIYPTNGYTRYDPKFQGRVTITADTSSNTAYMELSSLRSEDTAVYYCSRWGGDGFYAMDYWGQGTLVTVSS

SEQ ID NO: 54 - protein, 120 aa, Artificial Sequence
  Antibody heavy chain
EVQLVQSGPELVQPGGSLRLSCAASGFNIKDTYIHWVKQAPGKGLEWISRIYPTNGYTRYDPSFKGRATISADTSSNTAYLQVNSLRAEDTAVYYCSRWGGDGFYAMDYWGQGTLVTVSS

SEQ ID NO: 55 - protein, 5 aa, Artificial Sequence
  linker
GGGGG

SEQ ID NO: 56 - protein, 6 aa, Artificial Sequence
  linker
GSGSGS

SEQ ID NO: 57 - protein, 11 aa, Artificial Sequence
  linker
GSGGGTGGGSG

SEQ ID NO: 58 - protein, 3 aa, Artificial Sequence
  linker
PPP

SEQ ID NO: 59 - protein, 21 aa, Artificial Sequence
  linker
ASPAAPAPASPAAPAPSAPAA

SEQ ID NO: 60 - protein, 21 aa, Artificial Sequence
  linker
ASAAAPAAASAAASAPSAAAA

SEQ ID NO: 61 - protein, 25 aa, Artificial Sequence
  linker
AASPAAPSAPPAAASPAAPSAPPAA

SEQ ID NO: 62 - protein, 6 aa, Artificial Sequence
  linker
ASPASA

SEQ ID NO: 63 - protein, 9 aa, Artificial Sequence
  linker
ASPASPASA

SEQ ID NO: 64 - protein, 5 aa, Artificial Sequence
  linker
PAGSP

SEQ ID NO: 65 - protein, 5 aa, Artificial Sequence
  linker
STEPS

SEQ ID NO: 66 - protein, 5 aa, Artificial Sequence
  linker
STEEG

SEQ ID NO: 67 - protein, 5 aa, Artificial Sequence
  linker
GSAPG

SEQ ID NO: 68 - protein, 5 aa, Artificial Sequence
  linker
GASTP

SEQ ID NO: 69 - protein, 5 aa, Artificial Sequence
  linker
GPSAT

SEQ ID NO: 70 - protein, 220 aa, Artificial Sequence
  Antibody light chain
DIVMTQTPLSLSVTPGQPASISCKSSQSVFFRSNNKNILAWYLQKPGQPPQLLIYWASSRESGVPDRFSGSGSGTDFTLKISRVEAEDVGVYYCQQYFGSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 449 aa, Artificial Sequence
  Antibody heavy chain
EVQLVESGGGLVKPGGSLRLSCAASGFTFSSYSMSWVRQAPGKGLEWVSSISSSSSYIYYADSVKGRFTISRDNAKNSLYLQMNSLRAEDTAVYYCARGGDAYNYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 72 - protein, 217 aa, Artificial Sequence
  Antibody light chain
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 445 aa, Artificial Sequence
  Antibody heavy chain
EVQLVQSGAEVKKPGESLKISCKGSGYSFTSYWIGWVRQAPGQGLEWMGWISAYNGNTNYAQKLQGRVTMTTDTSTSTAYMELRSLRSDDTAVYYCAREGDGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 74 - protein, 214 aa, Artificial Sequence
  antibody light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 449 aa, Artificial Sequence
  Antibody heavy chain
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 76 - protein, 220 aa, Artificial Sequence; identical to SEQ ID NO: 11
  Antibody light chain

SEQ ID NO: 77 - protein, 448 aa, Artificial Sequence; identical to SEQ ID NO: 2
  Antibody heavy chain

SEQ ID NO: 78 - protein, 219 aa, Artificial Sequence
  Antibody light chain
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 446 aa, Artificial Sequence
  Antibody heavy chain
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 80 - protein, 448 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 81 - protein, 214 aa, Artificial Sequence
  Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 5 aa, Artificial Sequence
  linker
GGGGS

SEQ ID NO: 83 - protein, 10 aa, Artificial Sequence
  linker
GGGGSGGGGS

SEQ ID NO: 84 - protein, 15 aa, Artificial Sequence
  linker
GGGGSGGGGSGGGGS

SEQ ID NO: 85 - protein, 20 aa, Artificial Sequence
  linker
GGGGSGGGGSGGGGSGGGGS

SEQ ID NO: 86 - protein, 25 aa, Artificial Sequence
  linker
GGGGSGGGGSGGGGSGGGGSGGGGS

SEQ ID NO: 87 - protein, 7 aa, Artificial Sequence
  linker
GAAGAAG

SEQ ID NO: 88 - protein, 6 aa, Artificial Sequence
  linker
GGTGGT

SEQ ID NO: 89 - protein, 5 aa, Artificial Sequence
  linker
STSTS

SEQ ID NO: 90 - protein, 5 aa, Artificial Sequence
  linker
PAPAP

SEQ ID NO: 91 - protein, 7 aa, Artificial Sequence
  linker
SAASAAS

SEQ ID NO: 92 - protein, 447 aa, Artificial Sequence
  Antibody light chain
DIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 93 - protein, 699 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK